(19) United States
(12) Patent Application Publication
(10) Pub. No.: US 2019/0100725 A1
(43) Pub. Date: Apr. 4, 2019

(54) CELL CULTURE MEDIUM AND CULTURE MEDIUM SUPPLEMENT

(71) Applicant: Institute of Biophysics, Chinese Academy of Sciences, Beijing (CN)

(72) Inventors: Yue Ma, Beijing (CN); Junjie Jiang, Beijing (CN); Fei Pei, Beijing (CN)

(73) Assignee: Institute of Biophysics, Chinese Academy of Sciences, Beijing (CN)

(21) Appl. No.: 16/085,960

(22) PCT Filed: Mar. 18, 2016
(86) PCT No.: PCT/CN2016/076703

§ 371 Date: Sep. 17, 2018

(51) Int. Cl. C12N 5/00; C12N 5/077; C12N 5/074; C12N 5/0735

(52) U.S. Cl. CPC C12N 5/0037; C12N 5/0657; C12N 5/0696; C12N 5/0606; C12N 2501/155; C12N 2501/999; C12N 2506/45; C12N 2501/115; C12N 2500/38

(57) ABSTRACT

In one aspect, the present disclosure relates to a substantially albumin-free, chemically defined medium for efficiently supporting stem cell differentiation with significantly improved reproducibility and long-term culture of the differentiated cells. In particular embodiments, provided herein are compositions and methods for promoting atrial and ventricular cardiomyocytes formation from stem cells. The present disclosure further relates to the atrial and ventricular cardiomyocytes formed from the stem cells, and the uses of the cardiomyocytes, e.g., for cardiac injury repairmen, cardiac safety evaluation during drug discovery, and screening for new therapeutics for treating cardiac injuries.

### I. TECHNICAL FIELD

The present disclosure relates to compositions and methods for cell culture in general, for example, for culturing stem cells and enhancing differentiation efficiency of stem cells. In particular aspects, the present disclosure relates to compositions and methods for enhancing cardiac differentiation efficiency of stem cells and for promoting atrial and ventricular cardiomyocytes formation from stem cells, the atrial and ventricular cardiomyocytes formed from the stem cells, and the uses of the cardiomyocytes, e.g., for cardiac injury repair and screening for new therapeutics for treating cardiac injuries.

### II. BACKGROUND

Capable of almost unlimited proliferation and efficient differentiation into cardiomyocytes^{1, 2} human pluripotent stem cells (hPSCs) opened a new route for the research of human heart development and heart diseases, drug development, and most importantly, future heart cell replacement therapies for congestive heart failure^{3, 4}. To realize these prospects, particularly the clinical applications of these cells, a large number of cardiomyocytes, especially ventricular myocytes, has to be produced in a scalable and reproducible fashion in culture conditions that fulfill the bio-safety regulatory requirements for clinical applications⁵.

Current culture conditions for cardiac differentiation of hPSCs are one of the hurdles for safe therapeutic applications of hPSC-CMs. hPSCs were originally cultured on mouse embryonic fibroblast feeder cells with bovine serum replacement⁴. For cell-based transplantation therapies, using animal or human products in a cell culture system raises concerns of potential virus, mycoplasma and prion transmission to the cells. Comprehensive screening tests are required to exclude the risks of these infectious transmissions to the cell recipients, and satisfy the clinical regulatory requirements⁵. Most culture systems used today for cardiac differentiation contain animal products^{1, 2}. Recently, significant progress has been made in establishing chemically defined cardiac differentiation media with recombinant human albumin^{6, 7} However, beside the cost and batch variation of albumin, the large amounts of recombinant human albumin (5 mg/mL⁶ or 0.5 mg/mL⁷) in the medium may bring a significant amount of impurities into the culture. This raises the concern that these contaminating materials, if transplanted with the cells, could elicit an adverse immune response in the cell recipients⁸. Thus, there is need for improved compositions and methods to culture stem cells for clinical use.

### III. SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

To develop an albumin-free and chemically defined medium for cardiac differentiation, ingredients that either originate from animals (including albumin) or are unnecessary were systematically eliminated from B27, a wildly used medium supplement for hPSC cardiac differentiation^{2, 9}, and the medium was then supplemented with additional antioxidants to support efficient cell proliferation and cardiac differentiation. The statistics of the cardiac differentiation conducted over a period of 10 months indicated that the differentiation using this formulated albumin-free and chemically defined medium (S12 medium) was 20.9% higher in cardiac differentiation efficacy and 48.6% higher in cardiomyocyte yield compared with differentiations using B27-supplemented medium and with 57% reduction in inter-experimental variations. S12 medium supported not only large-scale production of cardiomyocytes in two-dimensional culture flasks, but also long-term culture of those cells (over 100 days). Using the E8 culture system and S12 medium, new hiPSC lines were derived, expanded, and differentiated into highly homogenous atrial- and ventricular-like cardiomyocyte populations in albumin-free and chemically defined culture environments. Electrophysiological studies demonstrated that the ventricular myocytes are suitable for drug cardiac safety analyses.

In one aspect, disclosed herein is a cell culture medium supplement, which comprises an antioxidant that substitutes the function of albumin in the cell culture medium. In one aspect, disclosed herein is a cell culture medium supplement, which comprises a combination of at least two antioxidants that substitute the function of albumin in the cell culture medium. In one aspect, disclosed herein is a cell culture medium supplement, which comprises a combination of at least three antioxidants that substitute the function of albumin in the cell culture medium.

In any of the preceding embodiments, the cell culture medium supplement disclosed herein can comprise at least one antioxidant selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof, wherein said cell culture medium supplement is configured to be combined with a basal culture medium to form a substantially albumin-free cell culture medium.

In any of the preceding embodiments, the cell culture medium supplement disclosed herein can comprise a combination of at least two antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof, wherein said cell culture medium supplement is configured to be combined with a basal culture medium to form a substantially albumin-free cell culture medium.

In any of the preceding embodiments, the cell culture medium supplement disclosed herein can comprise a combination of at least three different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof, wherein said cell culture medium supplement is configured to be combined with a basal culture medium to form a substantially albumin-free cell culture medium.

In any of the preceding embodiments, the cell culture medium supplement disclosed herein can comprise a combination of all four different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof.

In another aspect, disclosed herein is a substantially albumin-free cell culture medium, which comprises a substantially albumin-free basal culture medium and a cell culture medium supplement according to any of the preceding embodiments.

In any of the preceding embodiments, the cell culture medium can be configured to support growth and/or differentiation of a stem cell into a cardiomyocyte, e.g., a ventricular cardiomyocyte and/or an atrial cardiomyocyte. In any of the preceding embodiments, the cell culture medium can be configured to support maintenance of a cardiomyocyte, e.g., a ventricular cardiomyocyte and/or an atrial cardiomyocyte.

In another aspect, disclosed herein is a container which comprises a cell culture medium according to any of the preceding embodiments. In still another aspect, provided herein is a kit which comprises a cell culture medium according to any of the preceding embodiments.

In any of the preceding embodiments, the kit can further comprise a substance that initiates, directs and/or supports growth, differentiation, and/or maintenance of a cell. In any of the preceding embodiments, the kit can further comprise a substance that initiates, directs and/or supports differentiation and/or maintenance of a stem cell, a progenitor cell, or a precursor cell. In any of the preceding embodiments, the substance can initiate, direct, and/or support differentiation of a stem cell. In any of the preceding embodiments, the substance can initiate, direct, and/or support differentiation of a stem cell into a mesodermal cell. In any of the preceding embodiments, the substance can be a bone morphogenetic protein (BMP) antagonist. In any of the preceding embodiments, the BMP antagonist can be a BMP 4 antagonist. In any of the preceding embodiments, the substance can comprises basic fibroblast growth factor (bFGF), BMP 4, activin A, Wnt-3a or a small molecule which acts or functions like Wnt-3a (such as Bio and/or CHIR99021), and/or one or more growth factors and/or small molecules (e.g., dickkopf homolog 1 (DKK1), IWP, and inhibitor of Wnt response (IWR)) that inhibit the Wnt signaling pathway, or any suitable combination thereof.

In any of the preceding embodiments, the substance can initiate, direct and/or support differentiation of a stem cell or a mesodermal cell into a cardiomyocyte, e.g., a ventricular cardiomyocyte and/or an atrial cardiomyocyte.

In any of the preceding embodiments, the substance can initiate, direct and/or support differentiation of a stem cell or a mesodermal cell into a ventricular cardiomyocyte, wherein the substance can optionally comprise BMP 4 and/or an inhibitor of the retinoic acid signaling pathway. In any of the preceding embodiments, the substance can inhibit the retinoic acid signaling pathway, the SAPK/JNK signaling pathway, and/or the p38 signaling pathway in the stem cell or mesodermal cell. In any of the preceding embodiments, the substance can comprise a pan-retinoic acid receptor antagonist, a retinoic acid antagonist, a retinoic acid receptor antagonist, a retinoic X receptor antagonist, or a pan-retinoic acid receptor antagonist. In any of the preceding embodiments, the substance can comprise BMS-493, BMS-189453, SP-600125, and/or SB-203580.

In any of the preceding embodiments, the substance can initiate, direct and/or support differentiation of a stem cell or a mesodermal cell into an atrial cardiomyocyte. In any of the preceding embodiments, the substance can stimulate retinoic acid signaling pathway in the stem cell or mesodermal cell. In any of the preceding embodiments, the substance can comprise retinoic acid and/or vitamin A.

In any of the preceding embodiments, the kit can further comprise an instruction for supporting growth, differentiation and/or maintenance of a cell using the substantially albumin-free cell culture medium.

In yet another aspect, disclosed herein is a method for growing, differentiating and/or maintaining a cell, which method comprises contacting a cell with a substantially albumin-free cell culture medium according to any of the preceding embodiments. In one aspect, the method can be used to grow a cell. In another aspect, the method can be used to differentiate a cell. In still another aspect, the method can be used to maintain a cell.

In yet another aspect, disclosed herein is a cell grown, differentiated and/or maintained by the method according to any of the preceding embodiments. In another aspect, disclosed herein is a cardiomyocyte produced by the method according to any of the preceding embodiments. In one embodiment, a cardiomyocyte so produced has elevated expression level of a cardiomyocyte specific gene, embryonic cardiomyocyte-like action potentials (AP) and/or Cat²⁺ spark pattern typical of a cardiomyocyte.

In yet another aspect, disclosed herein is a ventricular cardiomyocyte produced by the method according to any of the preceding embodiments. In one embodiment, a ventricular cardiomyocyte so produced has elevated expression level of a ventricular specific gene, embryonic ventricular-like action potentials (AP) and/or Ca²⁺ spark pattern typical of a ventricular cardiomyocyte.

In still another aspect, disclosed herein is an atrial cardiomyocyte produced by the method according to any of the preceding embodiments. In one embodiment, an atrial cardiomyocyte so produced has embryonic atrial-like action potentials (AP) and/or Ca²⁺ spark pattern typical of an atrial cardiomyocyte.

In another aspect, disclosed herein is a pharmaceutical composition, which comprises an effective amount of the cells grown, differentiated and/or maintained by the method according to any of the preceding embodiments, and a pharmaceutically acceptable carrier or excipient.

In another aspect, disclosed herein is a pharmaceutical composition for treating a cardiac injury or disorder. In one aspect, the pharmaceutical composition comprises an effective amount of the cardiomyocytes, the ventricular cardiomyocytes, and/or the atrial cardiomyocytes generated according to any of the preceding embodiments, and a pharmaceutically acceptable carrier or excipient.

In one other aspect, disclosed herein is a method for treating a disease or disorder in a subject, which method comprises administering, to a subject to which such treatment is needed or desirable, an effective amount of the pharmaceutical composition according to any of the preceding embodiments.

In yet another aspect, disclosed herein is a method for treating a cardiac injury or disorder in a subject, which method comprises administering, to a subject to which such treatment is needed or desirable, an effective amount of the pharmaceutical composition according to any of the preceding embodiments.

### IV. BRIEF DESCRIPTION OF THE DRAWINGS

FIG. 1 shows cardiac differentiation medium optimization. (a) A schematic of hPSC cardiac differentiation, including the applied medium and the time points at which small molecules were applied. (b) Cell viability assay on cultures with B27 (RPMI 1640+B27), S12 (RPMI 1640+S12), S12 basal (RPMI 1640+S12 without antioxidants), or S12 basal-Cys-GSH (formulated RPMI 1640 with L-cysteine and glutathione depletion+S12 without antioxidants). (c) FACS analysis of the percentage of CTNT-positive cells in cultures incubated in S12 basal media supplemented with different combinations of antioxidants. (d) FACS analysis of the percentage of CTNT-positive cells, and (e) the yield of cardiomyocytes in cultures differentiated in S12 medium with the indicated compound withdrawal. (f) FACS analysis of the percentage of CTNT-positive cells cultured in S12 medium with different osmolarities. The statistics of the percentages of CTNT-positive cells (g), and the yields of the cardiomyocyte (h) of multiple cultures differentiated in RPMI 1640 medium supplemented with B27, S12 medium, or S12 basal medium (each spot represents an independent cardiac differentiation culture). Error bars, SD. n=3 independent biological replicates. Student's t-test, *P<0.05, ***P<0.001.

FIG. 2 shows direct generation of atrial and ventricular myocytes from hiPSCs. (a) FACS analysis of the percentage of CTNT-positive cells in five hiPSC lines differentiated in S12 medium. XVF: xeno and virus free. (b) Immunofluorescence staining of cardiac-related genes in 14-day-old differentiated cardiomyocytes. Scale bars, 100 μm. (c) RT-PCR analysis of the indicated genes during the differentiation process. (d) FACS analysis of the percentage of CTNT-positive cells in RA- or BMS493-treated cultures before (open bar) and after (gray bar) glucose deprivation enrichment by switching the energy source of the medium from glucose to lactate from day 12 to 14. (e) Quantitative PCR (QPCR) analysis of the expression levels of IRX4 and NR2F2 in differentially treated 14-day-old cultures. The average expression, normalized to GAPDH, is shown. (f) Immunofluorescence double staining of MLC2V and CTNT in 60-day-old RA- or BMS493-treated cultures. Scale bars, 100 μm. n=3 independent biological replicates. Error bars, SD. Student's t-test, *P<0.05, ***P<0.001.

FIG. 3 shows electrophysiological characterization of atrial- and ventricular-like cardiomyocytes. (a) Representative ventricular-like, atrial-like and nodal-like APs recorded from 30-day-old cardiomyocytes by whole-cell patch clamp. (b) The percentage of cardiomyocytes bearing ventricular-like, atrial-like and nodal-like APs in RA- and BMS493-treated cultures. (c) The parameters of the ventricular-like, atrial-like and nodal-like APs recorded in both RA- and BMS493-treated 30-day-old cultures. (d) Characteristics of the sodium current (I_{Na}) in cardiomyocytes treated with RA or BMS493. (e) Characteristics of the calcium current (I_{Ca}) in cardiomyocytes treated with RA or BMS493. (f) Characteristics of the E-4031-sensitive current (I_{Kr}) in cardiomyocyte cultures treated with RA or BMS493. (i) Representative I_{Kr} traces elicited by the protocol shown in the inset. 1 μM E-4031 was used to isolate the I_{Kr}. (ii) I-V plots of I_{Kr} at the end of the depolarizing step. (iii) Normalized tail current of I_{Kr}. (iv) Maximum tail current densities of I_{Kr}. Error bars, SEM.

FIG. 4 shows pharmacological responses of hPSC-derived ventricular-like cardiomyocytes. (a) The effects of DMSO treatment on evoked AP morphology of ventricular-like myocytes during the 20-min recording period. The dosage effects of E-4031 (b), nifedipine (c) and isoproterenol (d) on evoked AP morphologies of ventricular-like myocytes. The frequency of the stimulation was 1 Hz. The dashed lines indicate 0 mV.

FIG. 5 shows the expression profile of cardiac differentiation-related genes during the cardiac differentiation. QPCR analysis of the expression profile of a pluripotency marker gene (POU5F1), mesoderm marker gene (T, Brachyury), cardiac mesoderm marker gene (MESP1), committed cardiac progenitor marker genes (ISL1, NKX2.5 and TBX5) and cardiomyocyte marker genes (MLC2A and CTNT). The average expression, normalized to GAPDH, is shown. n=3 independent biological replicates. Error bars, SD.

FIG. 6 shows SDS-PAGE of 100 μL of RPMI 1640 medium supplemented with S12 and B27.

FIG. 7 shows large-scale cardiac differentiation. (a) Immunostaining of CTNT of 30-day-old culture in a T175 cell culture flask. Scale bars, 50 μm. (b) The cardiomyocyte yield per square centimeter of the 30-day-old cultures in 175 flasks, measured by FACS analysis of CTNT-positive cells. n=3 independent biological replicates. Error bars, SD.

FIG. 8 shows generation of hiPSCs in chemically defined conditions. (a) A schematic of the chemically defined generation of hiPSCs from human foreskin fibroblasts (HFF) with episomal plasmids expressing OCT4, SOX2, KLF4, L-MYC, p53 shRNA or Lin28. (b) Immunofluorescence staining of the pluripotency marker genes, NANOG, POU5F, SOX2, SSEA-4, TRA-1-60 and TRA-1-81 in newly generated hiPSC cells. Nuclei were stained with DAPI. Scale bar, 100 μm. (c) Hematoxylin and eosin (HE) staining of teratoma formed in NOD/SCID mice. Representative lineages of three germ lines, including endoderm (gut-like epithelium and respiratory epithelium), mesoderm (cartilage and muscle) and ectoderm (neural epithelium and epidermis), are indicated by arrows. Scale bar, 100 μm. (d) Karyotype of newly generated hiPSCs examined by chromosomal G-band analysis.

FIG. 9 shows early afterdepolarizations (EADs) induced by 100 nM E-4031 in spontaneously beating ventricular cells.

FIG. 10 shows an outline of the protocol used for the differentiation of hPSCs to cardiac lineages. D indicates the day of differentiation. From −D3 to D0, undifferentiated cells were cultured with E8 medium, and starting from D0, cardiac differentiation was induced with small molecules indicated in the chemical defined, albumin free medium. SAPK/JNK pathway or p38 MAPK small molecule inhibitors SP600125 or SB203580 were added to the culture between days 5 and 8.

FIG. 11 shows quantitative RT-PCR analysis of the kinetics of IRX4 and NR2F2 genes expression of cultures treated with different dose of SP600125 (FIG. 11A) and SB203580 (FIG. 11B). RA—retinoic acid treated cultures. B10-10 ng/ml BMP4 treated cultures. The average expression, normalized to GAPDH, is shown.

### V. DETAILED DESCRIPTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

All headings are for the convenience of the reader and should not be used to limit the meaning of the text that follows the heading, unless so specified.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

As used herein and in the appended claims, the singular forms “a,” “an,” and “the” include plural referents unless the context clearly dictates otherwise. For example, “a” or “an” means “at least one” or “one or more.” Thus, reference to “a stem cell” refers to one or more stem cells, and reference to “the method” includes reference to equivalent steps and methods disclosed herein and/or known to those skilled in the art, and so forth.

It is understood that aspects and embodiments of the disclosure described herein include “consisting” and/or “consisting essentially of” aspects and embodiments.

As used herein, “substantially albumin-free” may refer to that the cell culture medium or supplement comprises less than about 1×10⁻⁸ mg/mL, less than about 1×10⁻⁷ mg/mL, less than about 1×10⁶ mg/mL, less than about 1×10⁻⁵ mg/mL, less than about 1×10⁻⁴ mg/mL, less than about 1×10⁻³ mg/mL, less than about 0.01 mg/mL, less than about 0.1 mg/mL, less than about 1 mg/mL, less than about 3 mg/mL, or less than about 5 mg/mL albumin.

As used herein, “mammal” refers to any of the mammalian class of species. Frequently, the term “mammal,” as used herein, refers to humans, human subjects or human patients.

As used herein, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, “treatment” means any manner in which the symptoms of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, “production by recombinant means” refers to production methods that use recombinant nucleic acid methods that rely on well known methods of molecular biology for expressing proteins encoded by cloned nucleic acids.

As used herein, the term “subject” is not limited to a specific species or sample type. For example, the term “subject” may refer to a patient, and frequently a human patient. However, this term is not limited to humans and thus encompasses a variety of mammalian species.

As used herein, “pharmaceutically acceptable salts, esters or other derivatives” include any salts, esters or derivatives that may be readily prepared by those of skill in this art using known methods for such derivatization and that produce compounds that may be administered to animals or humans without substantial toxic effects and that either are pharmaceutically active or are prodrugs.

As used herein, a “prodrug” is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach. Oxford University Press, New York, pages 388-392).

As used herein, “test substance (or candidate compound)” refers to a chemically defined compound (e.g., organic molecules, inorganic molecules, organic/inorganic molecules, proteins, peptides, nucleic acids, oligonucleotides, lipids, polysaccharides, saccharides, or hybrids among these molecules such as glycoproteins, etc.) or mixtures of compounds (e.g., a library of test compounds, natural extracts or culture supernatants, etc.).

As used herein, high-throughput screening (HTS) refers to processes that test a large number of samples, such as samples of diverse chemical structures against disease targets to identify “hits” (see, e.g., Broach, et al., High throughput screening for drug discovery, Nature, 384:14-16 (1996); Janzen, et al., High throughput screening as a discovery tool in the pharmaceutical industry, Lab Robotics Automation: 8261-265 (1996); Fernandes, P. B., Letter from the society president, J. Biomol. Screening, 2:1 (1997); Burbaum, et al., New technologies for high-throughput screening, Curr. Opin. Chem. Biol., 1:72-78 (1997)). HTS operations are highly automated and computerized to handle sample preparation, assay procedures and the subsequent processing of large volumes of data.

### B. Cell Culture Medium Supplement

In one aspect, disclosed herein is a cell culture medium supplement, which comprises an antioxidant that substitutes the function(s) of albumin in the cell culture medium. In one aspect, disclosed herein is a cell culture medium supplement, which comprises a combination of at least two different antioxidants that substitute the function(s) of albumin in the cell culture medium. In some embodiments, the cell culture medium supplement can comprise 2, 3, 4, 5, 6, 7, 8, 9, 10 or more antioxidants that substitute the function(s) of albumin in the cell culture medium. The antioxidant(s) can be used to substitute any suitable function(s) of albumin, e.g., increasing the growth and productivity of cells and increase overall cell health, delivering important nutrients to cells, binding toxins to avoid toxic effects, binding excessive proteins to act as a buffer, binding hormones and growth peptides to keep them stable, and/or binding free radicals to reduce damage to cells. In some embodiments, the antioxidant(s) can be used to substitute any suitable levels of the function(s) of albumin, e.g., at least 50%, 60%, 70%, 80%, 90%, 95%, 99%, or 100% of the function(s) of albumin in the cell culture medium.

In one aspect, disclosed herein is a cell culture medium supplement comprising at least one antioxidant or at least two different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, d) pyruvic acid, pyruvate, or a salt or an ester thereof, e) a catalase, f) a superoxide dismutase, g) a thiol, such as 2-mercaptoethanol or 1-thioglycerol, h) a metallothione, i) a thioredoxin, j) lipoic acid or a salt or an ester thereof, k) uric acid or a salt or an ester thereof, 1) a carotene, m) melatonin, n) probucol, o) dimethylthiourea, and p) resveratrol, wherein said cell culture medium supplement is configured to be combined with a basal culture medium to form a substantially albumin-free cell culture medium.

In any of the preceding embodiments, the catalase can have a level ranging from about 0.025 μg/mL to about 250 μg/mL, e.g., about 2.5 μg/mL. In any of the preceding embodiments, the superoxide dismutase can have a level ranging from about 0.025 μg/mL to about 250 μg/mL, e.g., about 2.5 μg/mL. In any of the preceding embodiments, the 2-mercaptoethanol can have a level ranging from about 0.040 μg/mL to about 400 μg/mL, e.g., about 4 μg/mL. In any of the preceding embodiments, the 1-thioglycerol can have a level ranging from about 0.490 μg/mL to about 4900 μg/mL, e.g., about 49 μg/mL. In any of the preceding embodiments, the glutathione can have a level ranging from about 0.010 μg/mL to about 100 μg/mL, e.g., about 1 μg/mL. In any of the preceding embodiments, the metallothione can have a level ranging from about 0.010 μg/mL to about 100 μg/mL, e.g., about 1 μg/mL.

In any of the preceding embodiments, the cell culture medium supplement can comprise at least two different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof, wherein said cell culture medium supplement is configured to be combined with a basal culture medium to form a substantially albumin-free cell culture medium. In any of the preceding embodiments, the cell culture medium supplement can comprise at least three different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof. In any of the preceding embodiments, the cell culture medium supplement can comprise a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof.

In particular embodiments, the cell culture medium is substantially albumin-free when the cell culture medium comprises less than about 1×10⁻⁸ mg/mL, less than about 1×10⁻⁷ mg/mL, less than about 1×10⁻⁶ mg/mL, less than about 1×10⁻⁵ mg/mL, less than about 1×10⁻⁴ mg/mL, less than about 1×10⁻³ mg/mL, less than about 0.01 mg/mL, less than about 0.1 mg/mL, less than about 1 mg/mL, less than about 3 mg/mL, or less than about 5 mg/mL albumin.

In any of the preceding embodiments, the ascorbic acid, ascorbate, or a salt or an ester thereof, such as L-ascorbic acid, can have a level ranging from about 0.025 mg/mL to about 250 mg/mL, e.g., about 2.5 mg/mL. In particular embodiments, the level of the ascorbic acid, ascorbate, or a salt or an ester thereof, is between about 0.025 mg/mL and about 0.25 mg/mL, between about 0.25 mg/mL and about 2.5 mg/mL, between about 2.5 mg/mL and about 25 mg/mL, or between about 25 mg/mL and about 250 mg/mL.

In any of the preceding embodiments, the water-soluble analog of vitamin E can comprise trolox or MDL 73404 or a combination thereof.

In any of the preceding embodiments, the water-soluble analog of vitamin E, such as trolox, can have a level ranging from about 0.025 mM to about 250 mM, e.g., about 2.5 mM. In particular embodiments, the level of the water-soluble analog of vitamin E is between about 0.025 mM and about 0.25 mM, between about 0.25 mM and about 2.5 mM, between about 2.5 mM and about 25 mM, or between about 25 mM and about 250 mM.

In any of the preceding embodiments, the N-acetyl-cysteine or glutathione, or a salt or an ester thereof, such as N-acetyl-L-cysteine, can have a level ranging from about 0.025 mM to about 250 mM, e.g., about 2.5 mM. In particular embodiments, the level of the N-acetyl-cysteine or glutathione, or a salt or an ester thereof, is between about 0.025 mM and about 0.25 mM, between about 0.25 mM and about 2.5 mM, between about 2.5 mM and about 25 mM, or between about 25 mM and about 250 mM.

In any of the preceding embodiments, the pyruvic acid, pyruvate, or a salt or an ester thereof, such as sodium pyruvate, can have a level ranging from about 0.5 mM to about 5000 mM, e.g., about 50 mM. In particular embodiments, the level of the pyruvic acid, pyruvate, or a salt or an ester thereof, is between about 0.5 mM and about 5 mM, between about 5 mM and about 50 mM, between about 50 mM and about 500 mM, or between about 500 mM and about 5000 mM.

In any of the preceding embodiments, the cell culture medium supplement can further comprise an iron carrier.

In any of the preceding embodiments, the cell culture medium supplement can further comprise a polypeptide such as insulin and/or transferrin. In one aspect, the polypeptide is an iron carrier. In any of the preceding embodiments, the iron carrier can comprise Fe(III).

In any of the preceding embodiments, the cell culture medium supplement can comprise an iron carrier. In any of the preceding embodiments, the iron carrier can comprise Fe(III).

In any of the preceding embodiments, the iron carrier can be a transferrin or a Fe(III)-containing inorganic salt, such as Fe(NO₃)₃, iron(III) nitrate nonahydrate (Fe(NO₃)₃.9H₂O), or FeCl₃.

In any of the preceding embodiments, the polypeptide can comprise insulin. In any of the preceding embodiments, the polypeptide can comprise a mammalian polypeptide. In any of the preceding embodiments, the polypeptide can comprise a human polypeptide.

In any of the preceding embodiments, the polypeptide can be a recombinant polypeptide, which can be a recombinant human transferrin having a level ranging from about 0.0025 mg/mL to about 25 mg/mL, e.g., about 0.25 mg/mL, or can be a recombinant human insulin having a level ranging from about 0.002 mg/mL to about 20 mg/mL, e.g., about 0.2 mg/mL. In particular embodiments, the recombinant human transferrin level can be between about 0.0025 mg/mL and about 0.025 mg/mL, between about 0.025 mg/mL and about 0.25 mg/mL, between about 0.25 mg/mL and about 2.5 mg/mL, or between about 2.5 mg/mL and about 25 mg/mL. In particular embodiments, the recombinant human insulin level can be between about 0.002 mg/mL and about 0.02 mg/mL, between about 0.02 mg/mL and about 0.2 mg/mL, between about 0.2 mg/mL and about 2 mg/mL, or between about 2 mg/mL and about 20 mg/mL.

In any of the preceding embodiments, the polypeptide can have a level ranging from about 0.002 mg/mL to about 25 mg/mL, e.g., about 0.2 mg/mL or about 0.25 mg/mL. In particular embodiments, the level of the polypeptide can be between about 0.002 mg/mL and about 0.02 mg/mL, between about 0.02 mg/mL and about 0.2 mg/mL, between about 0.2 mg/mL and about 2 mg/mL, or between about 2 mg/mL and about 25 mg/mL.

In any of the preceding embodiments, the cell culture medium supplement can further comprise a water-soluble selenium compound. In some aspects, the selenium compound comprises sodium selenite (Na₂SeO₃), selenium dioxide (SeO₂), selenious acid (H₂SeO₃), seleninyl chloride (SeOCl₂), disodium selenate (Na₂SeO₄), or selenium sulfide (SeS), or any combination thereof.

In any of the preceding embodiments, the water-soluble selenium compound, such as sodium selenite (Na₂SeO₃), can have a level ranging from about 0.008 μg/mL to about 80 μg/mL, e.g., about 0.8 μg/mL. In particular embodiments, the level of the water-soluble selenium compound can be between about 0.008 μg/mL and about 0.08 μg/mL, between about 0.08 μg/mL and about 0.8 μg/mL, between about 0.8 μg/mL and about 8 μg/mL, or between about 8 μg/mL and about 80 μg/mL.

In any of the preceding embodiments, the cell culture medium supplement can further comprise a C₁₋₈ alkanolamine. In some aspects, the C₁₋₈ alkanolamine comprises ethanolamine, heptaminol, methanolamine, dimethylethanolamine, or N-methylethanolamine, or any combination thereof.

In any of the preceding embodiments, the C₁₋₈ alkanolamine, such as ethanolamine, can have a level ranging from about 0.0005 mg/mL to about 5 mg/mL, e.g., about 0.05 mg/mL. In particular embodiments, the level of the C₁₋₈ alkanolamine can be between about 0.0005 mg/mL and about 0.005 mg/mL, between about 0.005 mg/mL and about 0.05 mg/mL, between about 0.05 mg/mL and about 0.5 mg/mL, or between about 0.5 mg/mL and about 5 mg/mL.

In any of the preceding embodiments, the cell culture medium supplement can further comprise a C₁₋₈ quaternary ammonium compound. In some aspects, the C₁₋₈ quaternary ammonium compound comprises carnitine, tetraethylammonium bromide, tetramethylammonium chloride, tetramethylammonium hydroxide, or choline, or any combination thereof. In any of the preceding embodiments, the carnitine can be L-carnitine hydrochloride.

In any of the preceding embodiments, the C₁₋₈ quaternary ammonium compound, such as L-carnitine hydrochloride, can have a level ranging from about 0.001 mg/mL to about 10 mg/mL, e.g., 0.1 mg/mL. In particular embodiments, the level of the C₁₋₈ quaternary ammonium compound can be between about 0.001 mg/mL and about 0.01 mg/mL, between about 0.01 mg/mL to about 0.1 mg/mL, between about 0.1 mg/mL to about 1 mg/mL, or between about 1 mg/mL to about 10 mg/mL.

In any of the preceding embodiments, the cell culture medium supplement can further comprise a fatty acid, such as linoleic acid and linolenic acid or a combination thereof. In one aspect, the fatty acid can be in a solvent such as methyl-β-cyclodextrin.

In any of the preceding embodiments, the fatty acid can comprise a C₁₂₋₃₀ carbon chain and at least two double bonds. In any of the preceding embodiments, the fatty acid can comprise an 18-carbon chain and two or three double bonds. In any of the preceding embodiments, the fatty acid can comprise linolenic acid and/or linoleic acid.

In any of the preceding embodiments, the fatty acid, such as linoleic acid and linolenic acid or a combination thereof, can have a level ranging from about 0.0005 mg/mL to about 5 mg/mL, e.g., 0.05 mg/mL. In particular embodiments, the level of the fatty acid can be between about 0.0005 mg/mL and about 0.005 mg/mL, between about 0.005 mg/mL and about 0.05 mg/mL, between about 0.05 mg/mL and about 0.5 mg/mL, or between about 0.5 mg/mL and about 5 mg/mL.

In any of the preceding embodiments, the cell culture medium supplement can comprise: 1) ascorbic acid, ascorbate, or a salt or an ester thereof, 2) trolox, 3) N-acetyl-cysteine or glutathione, or a salt or an ester thereof, 4) pyruvic acid, pyruvate, or a salt or an ester thereof, 5) transferrin, 6) sodium selenite, 7) ethanolamine, 8) carnitine, 9) linolenic acid, and 10) linoleic acid.

In any of the preceding embodiments, the cell culture medium supplement can further comprise insulin. Therefore, provided herein are a cell culture medium supplement comprising insulin, and a cell culture medium supplement without insulin. In one aspect, the cell culture medium supplement without insulin is used for an early stage of the stem cell differentiation process.

In another aspect, disclosed herein is a container which comprises a cell culture medium supplement according to any of the preceding embodiments. In yet another aspect, provided herein is a kit which comprises a cell culture medium supplement according to any of the preceding embodiments. In one aspect, the kit further comprises an instruction for storing and/or using the cell culture medium supplement, e.g., for combining the cell culture medium supplement with a substantially albumin-free basal culture medium to prepare a substantially albumin-free cell culture medium.

In one aspect, the cell culture medium supplement can be used to enhance differentiation efficiency of any suitable stem cell. For example, the cell culture medium supplement can be used to enhancing cardiac differentiation efficiency of a totipotent, pluripotent, multipotent, oligopotent or unipotent stem cell. In another example, the cell culture medium supplement can be used to enhancing cardiac differentiation efficiency of an embryonic stem cell, an induced pluripotent stem cell, a fetal stem cell or an adult stem cell. In still another example, the cell culture medium supplement can be used to enhancing cardiac differentiation efficiency of a mammalian stem cell such as a human stem cell. In still another example, the cell culture medium supplement can be used to enhancing cardiac differentiation efficiency of a human embryonic stem cell or a human induced pluripotent stem cell.

### C. Cell Culture Medium

In one aspect, disclosed herein is a substantially albumin-free cell culture medium, which comprises a substantially albumin-free basal culture medium and a cell culture medium supplement according to any of the preceding embodiments.

In any of the preceding embodiments, in the cell culture medium, the level of the ascorbic acid, ascorbate, or a salt or an ester thereof, such as L-ascorbic acid, can range from about 0.5 mg/L to about 5000 mg/L, e.g., about 50 mg/L. In particular embodiments, the level of the ascorbic acid, ascorbate, or a salt or an ester thereof, is between about 0.5 mg/L and about 5 mg/L, between about 5 mg/L and about 50 mg/L, between about 50 mg/L and about 500 mg/L, or between about 500 mg/L and about 5000 mg/L.

In any of the preceding embodiments, in the cell culture medium, the level of a water-soluble analog of vitamin E, such as trolox, can range from about 0.5 μM to about 5000 μM, e.g., about 50 μM. In particular embodiments, the level of the water-soluble analog of vitamin E is between about 0.5 μM and about 5 μM, between about 5 μM and about 50 μM, between about 50 μM and about 500 μM, or between about 500 μM and about 5000 μM.

In any of the preceding embodiments, in the cell culture medium, the level of N-acetyl-cysteine or glutathione, or a salt or an ester thereof, such as N-acetyl-L-cysteine, can range from about 0.5 μM to about 5000 μM, e.g., about 50 μM. In particular embodiments, the level of N-acetyl-cysteine or glutathione, or a salt or an ester thereof, is between about 0.5 μM and about 5 μM, between about 5 μM and about 50 μM, between about 50 μM and about 500 μM, or between about 500 μM and about 5000 μM.

In any of the preceding embodiments, in the cell culture medium, the level of pyruvic acid, pyruvate, or a salt or an ester thereof, such as sodium pyruvate, can range from about 0.01 mM to about 100 mM, e.g., about 1 mM. In particular embodiments, the level of pyruvic acid, pyruvate, or a salt or an ester thereof, is between about 0.01 mM to about 0.1 mM, between about 0.1 mM to about 1 mM, between about 1 mM to about 10 mM, or between about 10 mM to about 100 mM.

In any of the preceding embodiments, in the cell culture medium, the level of the polypeptide, e.g., transferrin and/or insulin, can range from about 0.04 mg/L to about 500 mg/L, for example, the level of recombinant human transferrin can range from about 0.05 mg/L to about 500 mg/L, e.g., about 5 mg/L, and the level of recombinant human insulin can range from about 0.04 mg/L to about 400 mg/L, e.g., about 4 mg/L. In particular embodiments, the recombinant human transferrin level can be between about 0.05 mg/L and about 0.5 mg/L, between about 0.5 mg/L and about 5 mg/L, between about 5 mg/L and about 50 mg/L, or between about 50 mg/L and about 500 mg/L. In particular embodiments, the recombinant human insulin level can be between about 0.04 mg/L and about 0.4 mg/L, between about 0.4 mg/L and about 4 mg/L, between about 4 mg/L and about 40 mg/L, or between about 40 mg/mL and about 400 mg/L.

In any of the preceding embodiments, in the cell culture medium, the level of the selenium compound, e.g., sodium selenite (Na₂SeO₃), can range from about 0.16 pig/L to about 1600 μg/L, e.g., about 16 μg/L. In particular embodiments, the level of the selenium compound is between about 0.16 μg/L and about 1600 μg/L, between about 1.6 μg/L and about 16 μg/L, between about 16 μg/L and about 160 μg/L, or between about 160 μg/L and about 1600 μg/L.

In any of the preceding embodiments, in the cell culture medium, the level of the C₁₋₈ alkanolamine, e.g., ethanolamine, can range from about 0.01 mg/L to about 100 mg/L, e.g., about 1 mg/L. In particular embodiments, the level of the C₁₋₈ alkanolamine is between about 0.01 mg/L and about 0.1 mg/L, between about 0.1 mg/L and about 1 mg/L, between about 1 mg/L and about 10 mg/L, between about 10 mg/L and about 100 mg/L.

In any of the preceding embodiments, in the cell culture medium, the level of the C₁₋₈ quaternary ammonium compound, e.g., carnitine or L-carnitine hydrochloride, can range from about 0.02 mg/L to about 200 mg/L, e.g., 2 mg/L. In particular embodiments, the level of the C₁₋₈ quaternary ammonium compound is between about 0.02 mg/L and about 0.2 mg/L, between about 0.2 mg/L and about 2 mg/L, between about 2 mg/L and about 20 mg/L, between about 20 mg/L and about 200 mg/L.

In any of the preceding embodiments, in the cell culture medium, the level of the fatty acid, e.g., linolenic acid and/or linoleic acid, can range from about 0.01 mg/L to about 100 mg/L, e.g., 1 mg/L. In particular embodiments, the level of the fatty acid is between about 0.01 mg/L and about 0.1 mg/L, between about 0.1 mg/L and about 1 mg/L, between about 1 mg/L and about 10 mg/L, or between about 10 mg/L and about 100 mg/L.

In any of the preceding embodiments, in the cell culture medium, the substantially albumin-free basal culture medium can be selected from the group consisting of RPMI 1640, DMEM, DMEM/F12, IMDM, M199, and BME, or can be any suitable combination thereof.

In any of the preceding embodiments, in the cell culture medium, the ratio between the cell culture medium supplement and the substantially albumin-free basal culture medium can range from about 1:0.01 to about 1:100 (volume/volume), e.g., about 1:50 (volume/volume). In particular embodiments, the ratio is between about 1:0.01 to about 1:0.1, between about 1:0.1 to about 1:1, between about 1:1 to about 1:10, between about 1:10 to about 1:100, or smaller than about 1:100 (all ratios are volume/volume).

In any of the preceding embodiments, the cell culture medium can comprise 5 mg/mL or less albumin. In particular embodiments, the cell culture medium comprises less than about 1×10⁻⁸ mg/mL, less than about 1×10⁻⁷ mg/mL, less than about 1×10⁻⁶ mg/mL, less than about 1×10⁻⁵ mg/mL, less than about 1×10⁻⁴ mg/mL, less than about 1×10⁻³ mg/mL, less than about 0.01 mg/mL, less than about 0.1 mg/mL, less than about 1 mg/mL, or less than about 5 mg/mL albumin.

In any of the preceding embodiments, the cell culture medium can be configured to support growth, differentiation, and/or maintenance of a cell, such as a stem cell, a progenitor cell, or a precursor cell. In any of the preceding embodiments, the stem cell can be a totipotent, pluripotent, multipotent, oligopotent, or unipotent stem cell. In any of the preceding embodiments, the stem cell can be an embryonic stem cell, an induced pluripotent stem cell, a fetal stem cell, or an adult stem cell. In any of the preceding embodiments, the stem cell can be a mammalian stem cell. In any of the preceding embodiments, the mammalian stem cell can be a human stem cell. In any of the preceding embodiments, the stem cell can be a human embryonic stem cell or a human induced pluripotent stem cell.

In any of the preceding embodiments, the cell culture medium can be configured to support growth and/or differentiation of a stem cell into a cardiomyocyte, e.g., a ventricular cardiomyocyte and/or an atrial cardiomyocyte. In any of the preceding embodiments, the cell culture medium can be configured to support maintenance of a cardiomyocyte, e.g., a ventricular cardiomyocyte and/or an atrial cardiomyocyte.

In another aspect, disclosed herein is a container which comprises a cell culture medium according to any of the preceding embodiments. In still another aspect, provided herein is a kit which comprises a cell culture medium according to any of the preceding embodiments.

In any of the preceding embodiments, the kit can further comprise a substance that initiates, directs and/or supports growth, differentiation, and/or maintenance of a cell. In any of the preceding embodiments, the kit can further comprise a substance that initiates, directs and/or supports differentiation and/or maintenance of a stem cell, a progenitor cell, or a precursor cell. In any of the preceding embodiments, the substance can initiate, direct, and/or support differentiation of a stem cell. In any of the preceding embodiments, the substance can initiate, direct, and/or support differentiation of a stem cell into a mesodermal cell. In any of the preceding embodiments, the substance can be a bone morphogenetic protein (BMP) antagonist. In any of the preceding embodiments, the BMP antagonist can be a BMP 4 antagonist. In any of the preceding embodiments, the substance can comprises basic fibroblast growth factor (bFGF), BMP 4, activin A, Wnt-3a or a small molecule which acts or functions like Wnt-3a (such as Bio and/or CHIR99021), and/or one or more growth factors and/or small molecules (e.g., dickkopf homolog 1 (DKK1), IWP, and inhibitor of Wnt response (IWR)) that inhibit the Wnt signaling pathway, or any suitable combination thereof.

In any of the preceding embodiments, the substance can initiate, direct and/or support differentiation of a stem cell or a mesodermal cell into a cardiomyocyte, e.g., a ventricular cardiomyocyte and/or an atrial cardiomyocyte.

In any of the preceding embodiments, the substance can initiate, direct and/or support differentiation of a stem cell or a mesodermal cell into a ventricular cardiomyocyte, wherein the substance can optionally comprise BMP 4 and/or an inhibitor of the retinoic acid signaling pathway. In any of the preceding embodiments, the substance can inhibit the retinoic acid signaling pathway, the SAPK/JNK signaling pathway, and/or the p38 signaling pathway in the stem cell or mesodermal cell. In any of the preceding embodiments, the substance can comprise a pan-retinoic acid receptor antagonist, a retinoic acid antagonist, a retinoic acid receptor antagonist, a retinoic X receptor antagonist, or a pan-retinoic acid receptor antagonist. In any of the preceding embodiments, the substance can comprise BMS-493, BMS-189453, SP-600125, and/or SB-203580.

In any of the preceding embodiments, the substance can initiate, direct and/or support differentiation of a stem cell or a mesodermal cell into an atrial cardiomyocyte. In any of the preceding embodiments, the substance can stimulate retinoic acid signaling pathway in the stem cell or mesodermal cell. In any of the preceding embodiments, the substance can comprise retinoic acid and/or vitamin A.

In any of the preceding embodiments, the kit can further comprise an instruction for supporting growth, differentiation and/or maintenance of a cell using the substantially albumin-free cell culture medium.

In one aspect, the cell culture medium can be used to enhance differentiation efficiency of any suitable stem cell. For example, the cell culture medium can be used to enhancing cardiac differentiation efficiency of a totipotent, pluripotent, multipotent, oligopotent or unipotent stem cell. In another example, the cell culture medium can be used to enhancing cardiac differentiation efficiency of an embryonic stem cell, an induced pluripotent stem cell, a fetal stem cell or an adult stem cell. In still another example, the cell culture medium can be used to enhancing cardiac differentiation efficiency of a mammalian stem cell such as a human stem cell. In still another example, the cell culture medium can be used to enhancing cardiac differentiation efficiency of a human embryonic stem cell or a human induced pluripotent stem cell.

The stem cells can be obtained, prepared and/or maintained by any suitable methods. For example, mouse ES cells can grow on a layer of gelatin and require the presence of Leukemia Inhibitory Factor (LIF). Human ES cells can grow on a feeder layer of mouse embryonic fibroblasts (MEFs) and may require the presence of basic Fibroblast Growth Factor (bFGF or FGF-2). See e.g., Chambers et al., “Functional expression cloning of Nanog, a pluripotency sustaining factor in embryonic stem cells,” Cell 113 (5): 643-55 (2003).

A stem cell, e.g., a human embryonic stem cell, is often defined by the presence of several transcription factors and cell surface proteins. For example, the transcription factors Oct-4, Nanog, and Sox2 form the core regulatory network that ensures the suppression of genes that lead to differentiation and the maintenance of pluripotency. The cell surface antigens commonly used to identify hES cells are the glycolipids SSEA3 and SSEA4 and the keratan sulfate antigens Tra-1-60 and Tra-1-81.

Induced pluripotent stem cells, commonly abbreviated as iPS cells or iPSCs, are a type of pluripotent stem cell artificially derived from a non-pluripotent cell, typically an adult somatic cell, by inducing a “forced” expression of specific genes. Various genes, or a combination thereof, can be used to induce iPS cells from adult somatic cells. For example, Oct-3/4 and certain members of the Sox gene family (Sox1, Sox2, Sox3, and Sox15) can be used to induce iPS cells from adult somatic cells. Additional genes, including certain members of the Klf family (Klf1, Klf2, Klf4, and Klf5), the Myc family (C-myc, L-myc, and N-myc), Nanog, and LIN28, can be used to increase the induction efficiency. The various genes or its encoded proteins can be delivered into the adult somatic cells by any suitable methods. For example, various genes can be delivered into the adult somatic cell by a viral transfection system, such as a retroviral system, a lentiviral system, or an adenoviral system, or a plasmid without any virus transfection system. Alternatively, proteins encoded by the genes can be delivered into the adult somatic cells directly, e.g., by a repeated treatment of the cells with certain proteins channeled into the cells via poly-arginine anchors.

In one aspect, the stem cell can be induced to differentiae to form mesoderm by any suitable treatment or agent. In one example, the stem cell has differentiated to form mesoderm by contacting an undifferentiated stem cell with basic fibroblast growth factor (bFGF), BMP 4 and/or activin A. In another example, the stem cell has differentiated to form mesoderm by contacting an undifferentiated stem cell with basic fibroblast growth factor (bFGF), BMP 4 and activin A. The stem cell can be treated with bFGF, BMP 4 and activin A in any suitable order. For example, the stem cell can be differentiated to form mesoderm by contacting an undifferentiated stem cell with basic fibroblast growth factor (bFGF) and BMP 4 before the stem cell is contacted with activin A. In another example, the stem cell can be differentiated to form mesoderm by contacting an undifferentiated stem cell with Wnt-3a (Tran, et al., Wnt3a-induced mesoderm formation and cardiomyogenesis in human embryonic stem cells, Stem Cells 27: 1869-1878 (2009)), or a small molecule which acts or functions like Wnt-3a, such as Bio or CHIR99021.

Any suitable BMP antagonist can be used in the present methods to enhance cardiac differentiation efficiency of a stem cell. For example, a BMP 4 antagonist can be used. In another example, the BMP antagonist is Noggin. In still another example, the BMP antagonist is Chordin, Tsg, a member of DAN family (Yanagita, M. BMP antagonists: their roles in development and involvement in pathophysiology. Cytokine Growth Factor Rev 16: 309-317 (2005)), or a small molecule which acts or functions like BMP antagonist, such as Dorsomorphin (Hao, J. et al. Dorsomorphin, a selective small molecule inhibitor of BMP signaling, promotes cardiomyogenesis in embryonic stem cells. PLoS One 3: e2904 (2008)).

The present cell culture medium can further comprise a substance that inhibits retinoic acid signaling pathway in the stem cell. The retinoic acid signaling pathway in the stem cell can be inhibited by any suitable treatment or agent. In one example, the retinoic acid signaling pathway is inhibited by contacting the stem cell with a retinoic acid antagonist, a retinoic acid receptor antagonist or a retinoic X receptor antagonist. In another example, the retinoic acid signaling pathway is inhibited by contacting the stem cell with a pan-retinoic acid receptor antagonist, e.g., BMS-189453. In still another example, the retinoic acid signaling pathway is inhibited by contacting the stem cell with BMS-453, AGN194310, ANG193109, Ro41-5253, SR11335, 9-cis-retinoic acid, or a small molecule that inhibits retinoic acid synthesis, such as disulfiram and citral. In yet another example, the retinoic acid signaling pathway is inhibited by reducing or depleting vitamin A in the culture medium for the stem cell.

The present cell culture medium can further comprise a Wnt inhibitor to differentiate the stem cell into a cardiomyocyte. Any suitable Wnt inhibitor can be used. In one example, the Wnt inhibitor is dickkopf homolog 1 (DKK1).

### D. Methods and Compositions for Growing, Differentiating and/or Maintaining Cells

In yet another aspect, disclosed herein is a method for growing, differentiating and/or maintaining a cell, which method comprises contacting a cell with a substantially albumin-free cell culture medium according to any of the preceding embodiments. In one aspect, the method can be used to grow a cell. In another aspect, the method can be used to differentiate a cell. In still another aspect, the method can be used to maintain a cell.

In any of the preceding embodiments, the cell can be derived from a unicellular organism or a multicellular organism. In any of the preceding embodiments, the cell can be derived from a vertebrate, a non-human mammal or a human.

In any of the preceding embodiments, the cell can be a stem cell. In any of the preceding embodiments, the stem cell can be a totipotent, pluripotent, multipotent, oligopotent, or unipotent stem cell. In any of the preceding embodiments, the stem cell can be an embryonic stem cell, an induced pluripotent stem cell, a fetal stem cell, or an adult stem cell. In any of the preceding embodiments, the stem cell can be a mammalian stem cell. In any of the preceding embodiments, the mammalian stem cell can be a human stem cell. In any of the preceding embodiments, the stem cell can be a human embryonic stem cell or a human induced pluripotent stem cell.

In any of the preceding embodiments, a method disclosed herein can be used to support growth and/or differentiation of a stem cell into a cardiomyocyte, e.g., a ventricular cardiomyocyte and/or an atrial cardiomyocyte. In any of the preceding embodiments, the method can further comprise contacting a stem cell with a substance to initiate, direct and/or support differentiation of a stem cell into a mesodermal cell. In any of the preceding embodiments, the substance can be a bone morphogenetic protein (BMP) antagonist. In any of the preceding embodiments, the BMP antagonist can be a BMP 4 antagonist. In any of the preceding embodiments, the substance can comprise basic fibroblast growth factor (bFGF), BMP 4, activin A, Wnt-3a or a small molecule which acts or functions like Wnt-3a (such as Bio and/or CHIR99021), and/or one or more growth factors and/or small molecules (e.g., dickkopf homolog 1 (DKK1), IWP, and inhibitor of Wnt response (IWR)) that inhibit the Wnt signaling pathway, or any suitable combination thereof.

In any of the preceding embodiments, the method can further comprise contacting a stem cell or a mesodermal cell with a substance to initiate, direct and/or support differentiation of the stem cell or the mesodermal cell into a cardiomyocyte, e.g., a ventricular cardiomyocyte and/or an atrial cardiomyocyte. In any of the preceding embodiments, the substance can initiate, direct and/or support differentiation of a stem cell or a mesodermal cell into a ventricular cardiomyocyte.

In any of the preceding embodiments, the substance for initiating, directing, and/or supporting differentiation of the stem cell or the mesodermal cell can comprise BMP 4 and/or an inhibitor of the retinoic acid signaling pathway. In any of the preceding embodiments, the substance can inhibit the retinoic acid signaling pathway, the SAPK/JNK signaling pathway, and/or the p38 signaling pathway in the stem cell or mesodermal cell. In any of the preceding embodiments, the substance can comprise a pan-retinoic acid receptor antagonist, a retinoic acid antagonist, a retinoic acid receptor antagonist, a retinoic X receptor antagonist, or a pan-retinoic acid receptor antagonist, or a combination thereof. In any of the preceding embodiments, the substance can comprise BMS-493, BMS-189453, SP-600125, and/or SB-203580.

In another aspect, the substance used in a method disclosed herein can initiate, direct and/or support differentiation of a stem cell or a mesodermal cell into an atrial cardiomyocyte. In one aspect, the substance can stimulate retinoic acid signaling pathway in the stem cell or mesodermal cell. In any of the preceding embodiments, the substance can comprise retinoic acid and/or vitamin A.

In any of the preceding embodiments, the method can have a cardiac differentiation efficacy ranging from about 50% to about 90%, or more than about 90%. In particular embodiments, the method has a cardiac differentiation efficacy of about 50%, 55%, 600%, 65%, 70%, 75%/c, 80%, 85%, 90%, 95%, or 99%.

In any of the preceding embodiments, the method can be used to generate cardiomyocytes having an average density ranging from about 1×10⁵ cardiomyocytes/cm² to about 1×10⁶ cardiomyocytes/cm². In any of the preceding embodiments, the method can be used to generate cardiomyocytes having a yield ranging from about 1 cardiomyocyte to about 10 cardiomyocytes per stem cell, e.g., 1, 2, 3, 4, 5, 6, 7, 8. 9, or 10 cardiomyocyte(s) per stem cell. In other embodiments, the method has a yield of more than about 10 cardiomyocytes per stem cell.

In any of the preceding embodiments, the method can have a ventricular cardiac differentiation efficacy ranging from about 50% to about 90%, or more than about 900,%, for example, about 50⁰%, 55%, 60%, 65%, 70%, 75%, 80%, 85%, 90%, 95%, or 99%. In any of the preceding embodiments, the method can be used to generate ventricular cardiomyocytes having an average density ranging from about 1×10⁵ cardiomyocytes/cm² to about 1×10⁶ cardiomyocytes/cm². In any of the preceding embodiments, the method can be used to generate ventricular cardiomyocytes having a yield ranging from about 1 ventricular cardiomyocyte to about 10 ventricular cardiomyocytes per stem cell, e.g., 1, 2, 3, 4, 5, 6, 7, 8. 9, or 10 ventricular cardiomyocyte(s) per stem cell. In other embodiments, the method has a yield of more than about 10 ventricular cardiomyocytes per stem cell.

In any of the preceding embodiments, the method can have an atrial cardiac differentiation efficacy ranging from about 50%0 to about 90/o or more than about 90/o, for example, about 50%, 55%, 60%, 65%, 70%, 75%, 80%, 85%, 90%, 95%, or 99%. In any of the preceding embodiments, the method can be used to generate atrial cardiomyocytes having an average density ranging from about 1×10⁵ cardiomyocytes/cm² to about 1×10⁶ cardiomyocytes/cm². In any of the preceding embodiments, the method can be used to generate atrial cardiomyocytes having a yield ranging from about 1 atrial cardiomyocyte to about 10 atrial cardiomyocytes per stem cell, e.g., 1, 2, 3, 4, 5, 6, 7, 8. 9, or 10 atrial cardiomyocyte(s) per stem cell. In other embodiments, the method has a yield of more than about 10 atrial cardiomyocytes per stem cell.

In any of the preceding embodiments, the method can further comprise enriching the cardiomyocytes. In any of the preceding embodiments, the cardiomyocytes can be enriched by culturing cardiomyocytes in a medium with reduced glucose level. In some embodiments, the medium with reduced glucose level has a glucose level ranging from about 1000 mg/L to about 2000 mg/L. In some embodiments, the reduced glucose level ranges from about 1000 mg/L to about 1500 mg/L. In some embodiments, the reduced glucose level ranges from about 1500 mg/L to about 2000 mg/L. In some embodiments, the reduced glucose level is about 1500, 1600, 1700, 1800, 1900, or 2000 mg/L.

In any of the preceding embodiments, the method can be conducted at an osmolarity ranging from about 100 to about 500 mOsm. In particular embodiments, the method is conducted at an osmolarity ranging from about 100 to about 200 mOsm, from about 200 to about 300 mOsm, from about 300 to about 400 mOsm, or from about 400 to about 500 mOsm. In particular embodiments, the method is conducted at an osmolarity ranging from about 250 to about 350 mOsm, such as from about 300 to about 330 mOsm.

In yet another aspect, disclosed herein is a cell grown, differentiated and/or maintained by the method according to any of the preceding embodiments. In another aspect, disclosed herein is a cardiomyocyte produced by the method according to any of the preceding embodiments. In one embodiment, a cardiomyocyte so produced has elevated expression level of a cardiomyocyte specific gene, embryonic cardiomyocyte-like action potentials (AP) and/or Ca²⁺ spark pattern typical of a cardiomyocyte.

In yet another aspect, disclosed herein is a ventricular cardiomyocyte produced by the method according to any of the preceding embodiments. In one embodiment, a ventricular cardiomyocyte so produced has elevated expression level of a ventricular specific gene, embryonic ventricular-like action potentials (AP) and/or Ca²⁺ spark pattern typical of a ventricular cardiomyocyte.

In still another aspect, disclosed herein is an atrial cardiomyocyte produced by the method according to any of the preceding embodiments. In one embodiment, an atrial cardiomyocyte so produced has embryonic atrial-like action potentials (AP) and/or Ca²⁺ spark pattern typical of an atrial cardiomyocyte.

In another aspect, disclosed herein is a pharmaceutical composition, which comprises an effective amount of the cells grown, differentiated and/or maintained by the method according to any of the preceding embodiments, and a pharmaceutically acceptable carrier or excipient.

In another aspect, disclosed herein is a pharmaceutical composition for treating a cardiac injury or disorder. In one aspect, the pharmaceutical composition comprises an effective amount of the cardiomyocytes, the ventricular cardiomyocytes, and/or the atrial cardiomyocytes generated according to any of the preceding embodiments, and a pharmaceutically acceptable carrier or excipient.

In one other aspect, disclosed herein is a method for treating a disease or disorder in a subject, which method comprises administering, to a subject to which such treatment is needed or desirable, an effective amount of the pharmaceutical composition according to any of the preceding embodiments.

In yet another aspect, disclosed herein is a method for treating a cardiac injury or disorder in a subject, which method comprises administering, to a subject to which such treatment is needed or desirable, an effective amount of the pharmaceutical composition according to any of the preceding embodiments.

In any of the preceding embodiments, the method can be used for a subject who is a human.

In still another aspect, the present disclosure provides a method for promoting atrial cardiomyocyte formation from a stem cell, which method comprises stimulating or not inhibiting retinoic acid signaling pathway in a stem cell that has differentiated to form mesoderm.

The present methods can be used to promote atrial cardiomyocyte formation from any suitable stem cell. In one example, the present methods can be used to promote atrial cardiomyocyte formation from a totipotent, pluripotent, multipotent, oligopotent or unipotent stem cell. In another example, the present methods can be used to promote atrial cardiomyocyte formation from an embryonic stem cell, an induced pluripotent stem cell, a fetal stem cell or an adult stem cell. In still another example, the present methods can be used to promote atrial cardiomyocyte formation from a mammalian stem cell such as a human stem cell. In yet another example, the present methods can be used to promote atrial cardiomyocyte formation from a human embryonic stem cell or a human induced pluripotent stem cell.

The retinoic acid signaling pathway in the stem cell can be stimulated by any suitable treatment or agent. In one example, the retinoic acid signaling pathway in the stem cell is stimulated by contacting the stem cell with retinoic acid or vitamin A. In another example, the retinoic acid signaling pathway in the stem cell is stimulated by contacting the stem cell with a retinoic acid receptor agonist, such as LG100268 and LGD 1069.

### E. Pharmaceutical Compositions and Uses of the Cells

The cells, such as cardiomyocytes, can be used for any suitable purposes. In one aspect, the present disclosure provides a pharmaceutical composition for treating a cardiac injury or disorder, which pharmaceutical composition comprises an effective amount of the cells, such as cardiomyocytes, produced by the above methods, and optionally a pharmaceutically acceptable carrier or excipient. In one embodiment, the pharmaceutical composition comprises a mixture of atrial and ventricular cardiomyocytes. In another embodiment, the pharmaceutical composition comprises at least about 50%, preferably, at least about 60%, 70%, 80%, 90%, 95%, 99%, or 100% atrial cardiomyocytes. In still another embodiment, the pharmaceutical composition comprises at least about 50%, preferably, at least about 60%, 70%, 80%, 90%, 95%, 99%, or 100% ventricular cardiomyocytes.

In another aspect, the present disclosure provides a method for treating a cardiac injury or disorder in a subject, e.g., a human, which method comprises administering, to a subject to which such treatment is needed or desirable, an effective amount of the above pharmaceutical composition.

The formulation, dosage and route of administration of the cells, such as cardiomyocytes, whether predominantly atrial cardiomyocytes, predominantly ventricular cardiomyocytes or a mixture of atrial and ventricular cardiomyocytes, preferably in the form of pharmaceutical compositions, can be determined according to the methods known in the art (see e.g., Remington: The Science and Practice of Pharmacy. Alfonso R. Gennaro (Editor) Mack Publishing Company, April 1997; Therapeutic Peptides and Proteins: Formulation, Processing, and Delivery Systems, Banga, 1999; and Pharmaceutical Formulation Development of Peptides and Proteins, Hovgaard and Frkjr (Ed.), Taylor & Francis, Inc., 2000; Medical Applications of Liposomes, Lasic and Papahadjopoulos (Ed.), Elsevier Science, 1998; Textbook of Gene Therapy, Jain, Hogrefe & Huber Publishers, 1998; Adenoviruses: Basic Biology to Gene Therapy, Vol. 15, Seth, Landes Bioscience, 1999; Biopharmaceutical Drug Design and Development, Wu-Pong and Rojanasakul (Ed.), Humana Press, 1999; Therapeutic Angiogenesis: From Basic Science to the Clinic, Vol. 28, Dole et al. (Ed.), Springer-Verlag New York, 1999). In specific embodiments, the cardiomyocytes can be combined or formulated with endothelial cells, smooth muscle cells and/or fibroblast cells, and implanted into a heart. The cell or tissue patch can be transplanted by direct injection to the infarct area, injection with a catheter or implanted as a cardio-patch by a surgery. Preferably, the cardiomyocytes are formed from stem cells of the subject that is to be treated. Also preferably, the endothelial cells, smooth muscle cells and/or fibroblast cells are also obtained or derived from the subject that is to be treated, e.g., formed from stem cells of the subject that is to be treated.

The cardiomyocytes can be formulated for any suitable route of administration. In one example, the cardiomyocytes are administered by surgery or cell transplantation. The most suitable route in any given case will depend on the nature and severity of the condition being treated and on the nature of the particular cardiomyocytes which are being used.

The cardiomyocytes can be administered alone. Alternatively and preferably, the cardiomyocytes are co-administered with a pharmaceutically acceptable carrier or excipient. Any suitable pharmaceutically acceptable carrier or excipient can be used in the present method (See e.g., Remington: The Science and Practice of Pharmacy, Alfonso R. Gennaro (Editor) Mack Publishing Company, April 1997).

The present method can be used alone. Alternatively, the present method can be used in combination with other agent suitable for preventing, treating or delaying a cardiac injury, disease or disorder. Such other agent can be used before, with or after the administration of the cardiomyocytes. For example, the cardiomyocytes can be co-administered with such other agent.

According to the present invention, the cardiomyocytes, alone or in combination with other agents, carriers or excipients, may be formulated for any suitable administration route, such as surgery or cell transplantation. The method may employ formulations for administration in unit dosage form, in ampoules or in multidose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, sterile pyrogen-free water or other solvents, before use.

Pharmaceutically acceptable compositions and methods for their administration that may be employed for use in this invention include, but are not limited to those described in U.S. Pat. Nos. 5,736,154; 6,197,801 B1; 5,741,511; 5,886,039; 5,941,868; 6,258,374 B1; and 5,686,102.

The magnitude of a therapeutic dose in the treatment or prevention will vary with the severity of the condition to be treated and the route of administration. The dose, and perhaps dose frequency, will also vary according to age, body weight, condition and response of the individual patient.

It should be noted that the attending physician would know how to and when to terminate, interrupt or adjust therapy to lower dosage due to toxicity, or adverse effects. Conversely, the physician would also know how to and when to adjust treatment to higher levels if the clinical response is not adequate (precluding toxic side effects).

Any suitable route of administration may be used. Dosage forms include tablets, troches, cachet, dispersions, suspensions, solutions, capsules, patches, and the like. See, Remington's Pharmaceutical Sciences.

In practical use, the cardiomyocytes, alone or in combination with other agents, may be combined as the active in intimate admixture with a pharmaceutical carrier or excipient, such as beta-cyclodextrin and 2-hydroxy-propyl-beta-cyclodextrin, according to conventional pharmaceutical compounding techniques. The carrier may take a wide form of preparation desired for suitable administration. In preparing compositions for parenteral dosage form, such as intravenous injection or infusion, similar pharmaceutical media may be employed, water, glycols, oils, buffers, sugar, preservatives, liposomes, and the like known to those of skill in the art. Examples of such parenteral compositions include, but are not limited to dextrose 5% w/v, normal saline or other solutions. The total dose of the cardiomyocytes, alone or in combination with other agents to be administered may be administered in a vial of fluid, ranging from about 1×10³ to 1×10¹⁰ cells, e.g., 1×10³, 1×10⁴, 1×10⁵, 1×10⁶, 1×10⁷, 1×10⁸, 1×10⁹, or 1×10¹⁰ cells, or any subrange within the range of 1×10³ to 1×10¹⁰ cells.

The present disclosure also provides for kits for carrying out therapeutic regimens. Such kits comprise in one or more containers therapeutically effective amounts of the cardiomyocytes, alone or in combination with other agents, in pharmaceutically acceptable form. Preferred pharmaceutical forms would be in combination with sterile saline, dextrose solution, or buffered solution, or other pharmaceutically acceptable sterile fluid. Alternatively, the composition may be lyophilized or dessicated; in this instance, the kit optionally further comprises in a container a pharmaceutically acceptable solution, preferably sterile, to reconstitute the complex to form a solution for injection purposes. Exemplary pharmaceutically acceptable solutions are saline and dextrose solution.

In another embodiment, a kit of the invention further comprises a needle or syringe, preferably packaged in sterile form, for injecting the composition, and/or a packaged alcohol pad. Instructions are optionally included for administration of composition by a physician or by the patient.

### F. Other Uses of the Cells

The cells, such as cardiomyocytes, can be used for any suitable purposes.

In one aspect, disclosed herein is a method for identifying a modulator of a cell, and the method comprises: 1) contacting a cell grown, differentiated and/or maintained by the method according to any of the preceding embodiments with a modulator candidate and measuring the effect of the modulator candidate on a property of the cell; and 2) measuring the property of the cell not contacted with the modulator candidate. In embodiment, the property of the cell contacted with the modulator candidate is different from that of the cell not contacted with the modulator candidate identifies the modulator candidate as a modulator of the property of the cell.

In another aspect, disclosed herein is a method for identifying a modulator of a cardiomyocyte, and the method comprises: 1) contacting a cardiomyocyte, a ventricular cardiomyocyte, or an atrial cardiomyocyte generated according to any of the preceding embodiments with a modulator candidate and measuring the effect of the modulator candidate on a property of the cardiomyocyte; and 2) measuring the property of the cardiomyocyte not contacted with the modulator candidate. In embodiment, the property of the cardiomyocyte contacted with the modulator candidate is different from that of the cardiomyocyte not contacted with the modulator candidate identifies the modulator candidate as a modulator of the property of the cardiomyocyte.

The method can be conducted in any suitable format. Preferably, the method is conducted in a high-throughput screening (HTS) format.

Additional embodiments are provided herein to further illustrate the present disclosure.

### Embodiment 1

A cell culture medium supplement comprising at least one antioxidant or at least two different antioxidants that substitute(s) the function of albumin in a cell culture medium, wherein said cell culture medium supplement is configured to be combined with a basal culture medium to form a substantially albumin-free cell culture medium.

### Embodiment 2

A cell culture medium supplement comprising at least one antioxidant or at least two different antioxidants selected from the group consisting of: a) ascorbic acid, ascorbate, or a salt or an ester thereof, b) a water-soluble analog of vitamin E, c) N-acetyl-cysteine or glutathione, or a salt or an ester thereof, d) pyruvic acid, pyruvate, or a salt or an ester thereof, e) a catalase, f) a superoxide dismutase, g) a thiol, such as 2-mercaptoethanol or 1-thioglycerol, h) a metallothione, i) a thioredoxin, j) lipoic acid or a salt or an ester thereof, k) uric acid or a salt or an ester thereof, 1) a carotene, m) melatonin, n) probucol, o) dimethylthiourea, and p) resveratrol, wherein said cell culture medium supplement is configured to be combined with a basal culture medium to form a substantially albumin-free cell culture medium.

### Embodiment 3

The cell culture medium supplement of Embodiment 1 or Embodiment 2, which comprises at least one antioxidant or at least two, at least three, or all of the antioxidants, selected from the group consisting of: a) ascorbic acid, ascorbate, or a salt or an ester thereof, b) a water-soluble analog of vitamin E, c) N-acetyl-cysteine or glutathione, or a salt or an ester thereof, and d) pyruvic acid, pyruvate, or a salt or an ester thereof.

### Embodiment 4

The cell culture medium supplement of any of Embodiments 1-3, wherein the ascorbic acid, ascorbate, or a salt or an ester thereof, such as L-ascorbic acid, has a level ranging from about 0.025 mg/mL to about 250 mg/mL, e.g., about 2.5 mg/mL.

### Embodiment 5

The cell culture medium supplement of any of Embodiments 1-4, wherein the water-soluble analog of vitamin E is trolox or MDL 73404 or a combination thereof.

### Embodiment 6

The cell culture medium supplement of any of Embodiments 1-5, wherein the water-soluble analog of vitamin E, such as trolox, has a level ranging from about 0.025 mM to about 250 mM, e.g., about 2.5 mM.

### Embodiment 7

The cell culture medium supplement of any of Embodiments 1-6, wherein the N-acetyl-cysteine or glutathione, or a salt or an ester thereof, such as N-acetyl-L-cysteine, has a level ranging from about 0.025 mM to about 250 mM, e.g., about 2.5 mM.

### Embodiment 8

The cell culture medium supplement of any of Embodiments 1-7, wherein the pyruvic acid, pyruvate, or a salt or an ester thereof, such as sodium pyruvate, has a level ranging from about 0.5 mM to about 5000 mM, e.g., about 50 mM.

### Embodiment 9

The cell culture medium supplement of any of Embodiments 1-8, which further comprises an iron carrier.

### Embodiment 10

The cell culture medium supplement of any of Embodiments 1-9, which further comprises a polypeptide, which polypeptide is optionally the iron carrier.

### Embodiment 11

The cell culture medium supplement of Embodiment 9 or 10, wherein the iron carrier is a transferrin or a Fe(III)-containing inorganic salt such as Fe(NO₃)₃ and FeCl₃.

### Embodiment 12

The cell culture medium supplement of Embodiment 10 or 11, wherein the polypeptide is insulin or transferrin.

### Embodiment 13

The cell culture medium supplement of any of Embodiments 10-12, wherein the polypeptide is a mammalian polypeptide.

### Embodiment 14

The cell culture medium supplement of any of Embodiments 10-12, wherein the polypeptide is a human polypeptide.

### Embodiment 15

The cell culture medium supplement of any of Embodiments 10-14, wherein the polypeptide is a recombinant polypeptide, which is optionally a recombinant human transferrin having a level ranging from about 0.0025 mg/mL to about 25 mg/mL, e.g., about 0.25 mg/mL, or optionally a recombinant human insulin having a level ranging from about 0.002 mg/mL to about 20 mg/mL, e.g., about 0.2 mg/mL.

### Embodiment 16

The cell culture medium supplement of any of Embodiments 10-15, wherein the polypeptide has a level ranging from about 0.002 mg/mL to about 25 mg/mL, e.g., about 0.2 mg/mL or about 0.25 mg/mL.

### Embodiment 17

The cell culture medium supplement of any of Embodiments 1-16, which further comprises a water-soluble selenium compound.

### Embodiment 18

The cell culture medium supplement of Embodiment 17, wherein the selenium compound is sodium selenite (Na₂SeO₃), selenium dioxide (SeO₂), selenious acid (H₂SeO₃), seleninyl chloride (SeOCl₂), disodium selenate (Na₂SeO₄), or selenium sulfide (SeS), or any combination thereof.

### Embodiment 19

The cell culture medium supplement of Embodiment 17 or 18, wherein the water-soluble selenium compound, such as sodium selenite (Na₂SeO₃), has a level ranging from about 0.008 μg/mL to about 80 μg/mL, e.g., about 0.8 μg/mL.

### Embodiment 20

The cell culture medium supplement of any of Embodiments 1-19, which further comprises a C₁₋₈ alkanolamine.

### Embodiment 21

The cell culture medium supplement of Embodiment 20, wherein the C₁₋₈ alkanolamine is ethanolamine, heptaminol, methanolamine, dimethylethanolamine, or N-methylethanolamine, or any combination thereof.

### Embodiment 22

The cell culture medium supplement of Embodiment 20 or 21, wherein the C₁₋₈ alkanolamine, such as ethanolamine, has a level ranging from about 0.0005 mg/mL to about 5 mg/mL, e.g., about 0.05 mg/mL.

### Embodiment 23

The cell culture medium supplement of any of Embodiments 1-22, which further comprises a C₁₋₈ quaternary ammonium compound.

### Embodiment 24

The cell culture medium supplement of Embodiment 23, wherein the C₁₋₈ quaternary ammonium compound is carnitine, tetraethylammonium bromide, tetramethylammonium chloride, tetramethylammonium hydroxide, or choline, or any combination thereof.

### Embodiment 25

The cell culture medium supplement of Embodiment 24, wherein the carnitine is L-carnitine hydrochloride.

### Embodiment 26

The cell culture medium supplement of any of Embodiments 23-25, wherein the C₁₋₈ quaternary ammonium compound, such as L-carnitine hydrochloride, has a level ranging from about 0.001 mg/mL to about 10 mg/mL, e.g., 0.1 mg/mL.

### Embodiment 27

The cell culture medium supplement of any of Embodiments 1-26, which further comprises a fatty acid, such as linoleic acid and linolenic acid or a combination thereof, which fatty acid is optionally dissolved in the solvent methyl-3-cyclodextrin.

### Embodiment 28

The cell culture medium supplement of Embodiment 27, wherein the fatty acid comprises a C₁₂₋₃₀ carbon chain and at least two double bonds.

### Embodiment 29

The cell culture medium supplement of Embodiment 28, wherein the fatty acid comprises an 18-carbon chain and two or three double bonds.

### Embodiment 30

The cell culture medium supplement of Embodiment 28 or 29, wherein the fatty acid comprises linolenic acid and/or linoleic acid.

### Embodiment 31

The cell culture medium supplement of any of Embodiments 27-30, wherein the fatty acid, such as linoleic acid and linolenic acid or a combination thereof, has a level ranging from about 0.0005 mg/mL to about 5 mg/mL, e.g., 0.05 mg/mL.

### Embodiment 32

The cell culture medium supplement of any of Embodiments 1-31, which comprises: 1) ascorbic acid, ascorbate, or a salt or an ester thereof, 2) trolox, 3) N-acetyl-cysteine or glutathione, or a salt or an ester thereof, 4) pyruvic acid, pyruvate, or a salt or an ester thereof, 5) transferrin, 6) sodium selenite, 7) ethanolamine, 8) carnitine, 9) linolenic acid, and 10) linoleic acid.

### Embodiment 33

The cell culture medium supplement of Embodiment 32, which further comprises insulin.

### Embodiment 34

A container which comprises a cell culture medium supplement of any of Embodiments 1-33.

### Embodiment 35

A kit which comprises a cell culture medium supplement of any of Embodiments 1-33.

### Embodiment 36

The kit of Embodiment 35, which further comprises an instruction for storing and/or using the cell culture medium supplement, e.g., for combining the cell culture medium supplement with a substantially albumin-free basal culture medium to prepare a substantially albumin-free cell culture medium.

### Embodiment 37

A substantially albumin-free cell culture medium, which comprises a substantially albumin-free basal culture medium and a cell culture medium supplement of any of Embodiments 1-33.

### Embodiment 38

The cell culture medium of Embodiment 37, wherein the level of ascorbic acid, ascorbate, or a salt or an ester thereof, such as L-ascorbic acid, ranges from about 0.5 mg/L to about 5000 mg/L, e.g., about 50 mg/L.

### Embodiment 39

The cell culture medium of Embodiment 37 or 38, wherein the level of a water-soluble analog of vitamin E, such as trolox, ranges from about 0.5 μM to about 5000 μM, e.g., about 50 μM.

### Embodiment 40

The cell culture medium of any of Embodiments 37-39, wherein the level of N-acetyl-cysteine or glutathione, or a salt or an ester thereof, such as N-acetyl-L-cysteine, ranges from about 0.5 μM to about 5000 μM, e.g., about 50 μM.

### Embodiment 41

The cell culture medium of any of Embodiments 37-40, wherein the level of pyruvic acid, pyruvate, or a salt or an ester thereof, such as sodium pyruvate, ranges from about 0.01 mM to about 100 mM, e.g., about 1 mM.

### Embodiment 42

The cell culture medium of any of Embodiments 37-41, wherein the level of the polypeptide, e.g., transferrin and/or insulin, ranges from about 0.04 mg/L to about 500 mg/L, for example, the level of recombinant human transferrin can range from about 0.05 mg/L to about 500 mg/L, e.g., about 5 mg/L, and the level of recombinant human insulin can range from about 0.04 mg/L to about 400 mg/L, e.g., about 4 mg/L.

### Embodiment 43

The cell culture medium of any of Embodiments 37-42, wherein the level of the selenium compound, e.g., sodium selenite (Na₂SeO₃), ranges from about 0.16 μg/L to about 1600 μg/L, e.g., about 16 g/L.

### Embodiment 44

The cell culture medium of any of Embodiments 37-43, wherein the level of the C₁₋₈ alkanolamine, e.g., ethanolamine, ranges from about 0.01 mg/L to about 100 mg/L, e.g., about 1 mg/L.

### Embodiment 45

The cell culture medium of any of Embodiments 37-44, wherein the level of the C₁₋₈ quaternary ammonium compound, e.g., carnitine or L-carnitine hydrochloride, ranges from about 0.02 mg/L to about 200 mg/L, e.g., 2 mg/L.

### Embodiment 46

The cell culture medium of any of Embodiments 37-45, wherein the level of the fatty acid, e.g., linolenic acid and/or linoleic acid, ranges from about 0.01 mg/L to about 100 mg/L, e.g., 1 mg/L.

### Embodiment 47

The cell culture medium of any of Embodiments 37-46, wherein the substantially albumin-free basal culture medium is selected from the group consisting of RPMI 1640, DMEM, DMEM/F12, IMDM, M199, and BME.

### Embodiment 48

The cell culture medium of any of Embodiments 37-47, wherein the ratio between the cell culture medium supplement and the substantially albumin-free basal culture medium ranges from about 1:0.01 to about 1:100 (volume/volume), e.g., about 1:50 (volume/volume).

### Embodiment 49

The cell culture medium of any of Embodiments 37-48, which comprises 5 mg/mL or less albumin.

### Embodiment 50

The cell culture medium of any of Embodiments 37-49, which is configured to support growth, differentiation, and/or maintenance of a cell.

### Embodiment 51

The cell culture medium of Embodiment 50, which is configured to support growth, differentiation and/or maintenance of a stem cell, a progenitor cell, or a precursor cell.

### Embodiment 52

The cell culture medium of Embodiment 51, wherein the stem cell is a totipotent, pluripotent, multipotent, oligopotent, or unipotent stem cell.

### Embodiment 53

The cell culture medium of Embodiment 51, wherein the stem cell is an embryonic stem cell, an induced pluripotent stem cell, a fetal stem cell, or an adult stem cell.

### Embodiment 54

The cell culture medium of Embodiment 51, wherein the stem cell is a mammalian stem cell.

### Embodiment 55

The cell culture medium of Embodiment 54, wherein the mammalian stem cell is a human stem cell.

### Embodiment 56

The cell culture medium of Embodiment 51, wherein the stem cell is a human embryonic stem cell or a human induced pluripotent stem cell.

### Embodiment 57

The cell culture medium of any of Embodiments 51-56, which is configured to support growth and/or differentiation of a stem cell into a cardiomyocyte, e.g., a ventricular cardiomyocyte and/or an atrial cardiomyocyte.

### Embodiment 58

The cell culture medium of any of Embodiments 51-56, which is configured to support maintenance of a cardiomyocyte, e.g., a ventricular cardiomyocyte and/or an atrial cardiomyocyte.

### Embodiment 59

A container which comprises a cell culture medium of any of Embodiments 37-58.

### Embodiment 60

A kit which comprises a cell culture medium of any of Embodiments 37-58.

### Embodiment 61

The kit of Embodiment 60, which further comprises a substance that initiates, directs and/or supports growth, differentiation, and/or maintenance of a cell.

### Embodiment 62

The kit of Embodiment 60, which further comprises a substance that initiates, directs and/or supports differentiation and/or maintenance of a stem cell, a progenitor cell, or a precursor cell.

### Embodiment 63

The kit of Embodiment 62, wherein the substance initiates, directs, and/or supports differentiation of a stem cell.

### Embodiment 64

The kit of Embodiment 63, wherein the substance initiates, directs and/or supports differentiation of a stem cell into a mesodermal cell.

### Embodiment 65

The kit of Embodiment 63, wherein the substance is a bone morphogenetic protein (BMP) antagonist.

### Embodiment 66

The kit of Embodiment 65, wherein the BMP antagonist is a BMP 4 antagonist.

### Embodiment 67

The kit of Embodiment 63, wherein the substance comprises basic fibroblast growth factor (bFGF), BMP 4, activin A, Wnt-3a or a small molecule which acts or functions like Wnt-3a (such as Bio and/or CHIR99021), and/or one or more growth factors and/or small molecules (e.g., dickkopf homolog 1 (DKK1), IWP, and inhibitor of Wnt response (IWR)) that inhibit the Wnt signaling pathway.

### Embodiment 68

The kit of Embodiment 63, wherein the substance initiates, directs and/or supports differentiation of a stem cell or a mesodermal cell into a cardiomyocyte, e.g., a ventricular cardiomyocyte and/or an atrial cardiomyocyte.

### Embodiment 69

The kit of Embodiment 68, wherein the substance initiates, directs and/or supports differentiation of a stem cell or a mesodermal cell into a ventricular cardiomyocyte, wherein the substance optionally comprises BMP 4 and/or an inhibitor of the retinoic acid signaling pathway.

### Embodiment 70

The kit of Embodiment 69, wherein the substance inhibits the retinoic acid signaling pathway, the SAPK/JNK signaling pathway, and/or the p38 signaling pathway in the stem cell or mesodermal cell.

### Embodiment 71

The kit of Embodiment 70, wherein the substance is a pan-retinoic acid receptor antagonist, a retinoic acid antagonist, a retinoic acid receptor antagonist, a retinoic X receptor antagonist, or a pan-retinoic acid receptor antagonist.

### Embodiment 72

The kit of Embodiment 70, wherein the substance is BMS-493, BMS-189453, SP-600125, or SB-203580.

### Embodiment 73

The kit of Embodiment 68, wherein the substance initiates, directs and/or supports differentiation of a stem cell or a mesodermal cell into an atrial cardiomyocyte.

### Embodiment 74

The kit of Embodiment 73, wherein the substance stimulates retinoic acid signaling pathway in the stem cell or mesodermal cell.

### Embodiment 75

The kit of Embodiment 74, wherein the substance is retinoic acid or vitamin A.

### Embodiment 76

The kit of any of Embodiments 60-75, which further comprises an instruction for supporting growth, differentiation and/or maintenance of a cell (such as a stem cell, a progenitor cell, or a precursor cell) using the substantially albumin-free cell culture medium.

### Embodiment 77

A method for growing, differentiating and/or maintaining a cell, which method comprises contacting a cell with a substantially albumin-free cell culture medium of any one of Embodiments 37-58.

### Embodiment 78

The method of Embodiment 77, which is used to grow a cell.

### Embodiment 79

The method of Embodiment 77, which is used to differentiate a cell.

### Embodiment 80

The method of Embodiment 77, which is used to maintain a cell.

### Embodiment 81

The method of any of Embodiments 77-80, wherein the cell is derived from a unicellular organism or a multicellular organism.

### Embodiment 82

The method of any of Embodiments 77-80, wherein the cell is derived from a vertebrate, a non-human mammal or a human.

### Embodiment 83

The method of any of Embodiments 77-82, wherein the cell is a stem cell.

### Embodiment 84

The method of Embodiment 83, wherein the stem cell is a totipotent, pluripotent, multipotent, oligopotent or unipotent stem cell.

### Embodiment 85

The method of Embodiment 83, wherein the stem cell is an embryonic stem cell, an induced pluripotent stem cell, a fetal stem cell or an adult stem cell.

### Embodiment 86

The method of Embodiment 83, wherein the stem cell is a mammalian stem cell.

### Embodiment 87

The method of Embodiment 86, wherein the mammalian stem cell is a human stem cell.

### Embodiment 88

The method of Embodiment 83, wherein the stem cell is a human embryonic stem cell or a human induced pluripotent stem cell.

### Embodiment 89

The method of any of Embodiments 83-88, which is used to support growth and/or differentiation of a stem cell into a cardiomyocyte, e.g., a ventricular cardiomyocyte and/or an atrial cardiomyocyte.

### Embodiment 90

The method of any of Embodiments 83-89, which further comprises contacting a stem cell with a substance to initiate, direct and/or support differentiation of a stem cell into a mesodermal cell.

### Embodiment 91

The method of Embodiment 90, wherein the substance is a bone morphogenetic protein (BMP) antagonist.

### Embodiment 92

The method of Embodiment 91, wherein the BMP antagonist is a BMP 4 antagonist.

### Embodiment 93

The method of Embodiment 91, wherein the substance comprises basic fibroblast growth factor (bFGF), BMP 4, activin A, Wnt-3a or a small molecule which acts or functions like Wnt-3a (such as Bio and/or CHIR99021), and/or one or more growth factors and/or small molecules (e.g., dickkopf homolog 1 (DKK1), IWP, and inhibitor of Wnt response (IWR)) that inhibit the Wnt signaling pathway.

### Embodiment 94

The method of any of Embodiments 83-93, which further comprises contacting a stem cell or a mesodermal cell with a substance to initiate, direct and/or support differentiation of the stem cell or the mesodermal cell into a cardiomyocyte, e.g., a ventricular cardiomyocyte and/or an atrial cardiomyocyte.

### Embodiment 95

The method of Embodiment 94, wherein the substance initiates, directs and/or supports differentiation of a stem cell or a mesodermal cell into a ventricular cardiomyocyte, wherein the substance optionally comprises BMP 4 and/or an inhibitor of the retinoic acid signaling pathway.

### Embodiment 96

The method of Embodiment 95, wherein the substance inhibits the retinoic acid signaling pathway, the SAPK/JNK signaling pathway, and/or the p38 signaling pathway in the stem cell or mesodermal cell.

### Embodiment 97

The method of Embodiment 96, wherein the substance is a pan-retinoic acid receptor antagonist, a retinoic acid antagonist, a retinoic acid receptor antagonist, a retinoic X receptor antagonist, or a pan-retinoic acid receptor antagonist.

### Embodiment 98

The method of Embodiment 97, wherein the substance is BMS-493, BMS-189453, SP-600125, or SB-203580.

### Embodiment 99

The method of Embodiment 95, wherein the substance initiates, directs and/or supports differentiation of a stem cell or a mesodermal cell into an atrial cardiomyocyte.

### Embodiment 100

The method of Embodiment 99, wherein the substance stimulates retinoic acid signaling pathway in the stem cell or mesodermal cell.

### Embodiment 101

The method of Embodiment 100, wherein the substance is retinoic acid or vitamin A.

### Embodiment 102

The method of any of Embodiments 94-101, which has a cardiac differentiation efficacy ranging from about 50% to about 90%.

### Embodiment 103

The method of any of Embodiments 94-102, which generates cardiomyocytes having an average density ranging from about 1×10⁵ cardiomyocytes/cm² to about 1×10⁶ cardiomyocytes/cm².

### Embodiment 104

The method of any of Embodiments 95-98, 102, and 103, which generates cardiomyocytes having a yield ranging from about 1 cardiomyocyte to about 10 cardiomyocytes per stem cell.

### Embodiment 105

The method of any of Embodiments 95-98 and 102-104, which has a ventricular cardiac differentiation efficacy ranging from about 50% to about 90%.

### Embodiment 106

The method of any of Embodiments 95-98 and 102-105, which generates ventricular cardiomyocytes having an average density ranging from about 1×10⁵ cardiomyocytes/cm² to about 1×10⁶ cardiomyocytes/cm².

### Embodiment 107

The method of any of Embodiments 95-98 and 102-106, which generates ventricular cardiomyocytes having a yield ranging from about 1 cardiomyocyte to about 10 cardiomyocytes per stem cell.

### Embodiment 108

The method of any of Embodiments 99-104, which has an atrial cardiac differentiation efficacy ranging from about 50% to about 90%.

### Embodiment 109

The method of any of Embodiments 99-104 and 108, which generates atrial cardiomyocytes having an average density ranging from about 1×10⁵ cardiomyocytes/cm² to about 1×10⁶ cardiomyocytes/cm².

### Embodiment 110

The method of any of Embodiments 99-104, 108, and 109, which generates atrial cardiomyocytes having a yield ranging from about 1 cardiomyocyte to about 10 cardiomyocytes per stem cell.

### Embodiment 111

The method of any of Embodiments 94-110, which further comprises enriching the cardiomyocytes.

### Embodiment 112

The method of Embodiment 111, wherein the cardiomyocytes are enriched by culturing cardiomyocytes in a medium with a reduced glucose level, wherein the reduced glucose level ranges from about 1000 mg/L to about 2000 mg/L.

### Embodiment 113

The method of any of Embodiments 77-112, which is conducted at an osmolarity ranging from about 100 to about 500 mOsm, e.g., 100-200 mOsm, 200-300 mOsm, 250-350 mOsm (such as 300-330 mOsm), 300-400 mOsm, and 400-500 mOsm.

### Embodiment 114

A cell grown, differentiated and/or maintained by the method of any of Embodiments 77-113.

### Embodiment 115

A cardiomyocyte produced by the method of any of Embodiments 94-113.

### Embodiment 116

A cardiomyocyte of Embodiment 115, which has elevated expression level of a cardiomyocyte specific gene, embryonic cardiomyocyte-like action potentials (AP) and/or Ca²⁺ spark pattern typical of a cardiomyocyte.

### Embodiment 117

A ventricular cardiomyocyte produced by the method of any of Embodiment 94-98, 102-107, and 111-113.

### Embodiment 118

A ventricular cardiomyocyte of Embodiment 117, which has elevated expression level of a ventricular specific gene, embryonic ventricular-like action potentials (AP) and/or Ca²⁺ spark pattern typical of a ventricular cardiomyocyte.

### Embodiment 119

An atrial cardiomyocyte produced by the method of any of Embodiments 94, 99-104, and 108-113.

### Embodiment 120

An atrial cardiomyocyte of Embodiment 119, which has embryonic atrial-like action potentials (AP) and/or Ca²⁺, spark pattern typical of an atrial cardiomyocyte.

### Embodiment 121

A pharmaceutical composition, which pharmaceutical composition comprises an effective amount of the cells grown, differentiated and/or maintained by the method of any of Embodiments 77-113, and a pharmaceutically acceptable carrier or excipient.

### Embodiment 122

A pharmaceutical composition for treating a cardiac injury or disorder, which pharmaceutical composition comprises an effective amount of the cardiomyocytes of Embodiment 115 or 116, ventricular cardiomyocytes of Embodiment 117 or 118, or atrial cardiomyocytes of Embodiment 119 or 120, and a pharmaceutically acceptable carrier or excipient.

### Embodiment 123

A method for treating a disease or disorder in a subject, which method comprises administering, to a subject to which such treatment is needed or desirable, an effective amount of the pharmaceutical composition of Embodiment 121.

### Embodiment 124

A method for treating a cardiac injury or disorder in a subject, which method comprises administering, to a subject to which such treatment is needed or desirable, an effective amount of the pharmaceutical composition of Embodiment 122.

### Embodiment 125

The method of Embodiment 123 or 124, wherein the subject is a human.

### Embodiment 126

A method for identifying a modulator of a cell, which method comprises: 1) contacting a cell of Embodiment 114 with a modulator candidate and measuring the effect of the modulator candidate on a property of the cell; and 2) measuring the property of the cell not contacted with the modulator candidate, whereby the property of the cell contacted with the modulator candidate is different from that of the cell not contacted with the modulator candidate identifies the modulator candidate as a modulator of the property of the cell.

### Embodiment 127

A method for identifying a modulator of a cardiomyocyte, which method comprises: 1) contacting a cardiomyocyte of Embodiment 115 or 116, a ventricular cardiomyocyte of Embodiment 117 or 118, or an atrial cardiomyocyte of Embodiment 119 or 120 with a modulator candidate and measuring the effect of the modulator candidate on a property of the cardiomyocyte; and 2) measuring the property of the cardiomyocyte not contacted with the modulator candidate, whereby the property of the cardiomyocyte contacted with the modulator candidate is different from that of the cardiomyocyte not contacted with the modulator candidate identifies the modulator candidate as a modulator of the property of the cardiomyocyte.

### G. Examples

### Example 1

### Chemically Defined Albumin-Free Generation of Human Atrial and Ventricular Cardiomyocytes

Overview

All existing culture media for cardiac differentiation of human pluripotent stem cells (hPSCs) contain significant amounts of albumin. For clinical applications of hPSC-derived cardiomyocytes (hPSC-CMs), albumin raises the concern of immunogenicity. Its batch variation is one of the major causes of inconsistent hPSC cardiac differentiation. In this example, it is demonstrated that the antioxidants, such as L-ascorbic acid, trolox, N-acetyl-L-cysteine, and pyruvate (such as sodium pyruvate), could functionally substitute albumin in the culture medium. With this substitution, an albumin-free, chemically defined medium (S12 medium) was formulated, and the medium efficiently supported hPSC cardiac differentiation with significantly improved reproducibility, and long-term culture of hPSC-CMs. Under chemically defined and albumin-free conditions, human induced pluripotent stem cells (hiPSCs) were established, and directed to differentiate into highly homogenous atrial and ventricular myocytes, and their electrophysiological properties were characterized. Finally, it is demonstrated that hiPSC-derived ventricular myocytes generated in this culture system were suitable for cardiac safety evaluation for drug discovery. In summary, this simplified, chemically defined culture medium should facilitate both research and clinical applications of hPSC-CMs.

Material and Methods

Human Induced Pluripotent Cell Derivation.

With approved protocols by the Review Board of the Institute of Biophysics and a written consent obtained from the patient, foreskin tissue was obtained from a 5-year-old boy after circumcision surgery. The tissue was cut into small pieces and incubated in TrypLE Select enzyme solution (Life Technology, 0040090DG) at 4° C. overnight, and at 37° C. for 30 min. Small pieces of tissue were washed three times with E6 medium (E8 medium without bFGF and TGF-β) and plated in a 24-well plate pre-coated with recombinant human truncated vitronectin (VTN-NC, 2.2 μg/cm²). The cells were cultured in E8 medium supplemented with 100 ng/mL recombinant human EGF (Peprotech, AF-100-15)¹⁰ for 4-5 days to obtain fibroblast outgrowth. To reprogram the fibroblasts, 1.25 μg each of the episomal plasmids (Addgene), pCXLE-hOCT3/4-shp53 (27077), pCXLE-hSK (27078) and pCXLE-hUL (27080), were electroporated together into 1×10⁶ foreskin fibroblasts (passage number<6) using an Amaxa apparatus, with the setting: program U-018, and Nucleofector kit VPD-1001 (Lonza). The cells were then plated in a vitronectin-coated plate. E8 plus hydrocortisone medium were used before the cells reached ˜20% confluence, then the medium was changed to regular E8 medium. Colonies with hESC morphology were picked into a 24-well plate (one colony per well) pre-coated with VTN-NC and cultured in E8 medium containing 10 μM Y27632 (Abcam, ab120129) for the first 24 h. The hiPSCs clones were then digested with Versene solution (Life Technology, 21051-024, 15040-066) for culture expansion^{10, 18}.

Maintenance and Differentiation of hPSCs.

To maintain hPSCs, hESC line H7 (WiCell Research Institute) and hiPSC lines were cultured in a humidified incubator with 5% CO₂ at 37° C. Undifferentiated hPSCs were routinely maintained on 2.2 μg/cm² VTN-NC-coated plates with E8 medium as described¹⁰.

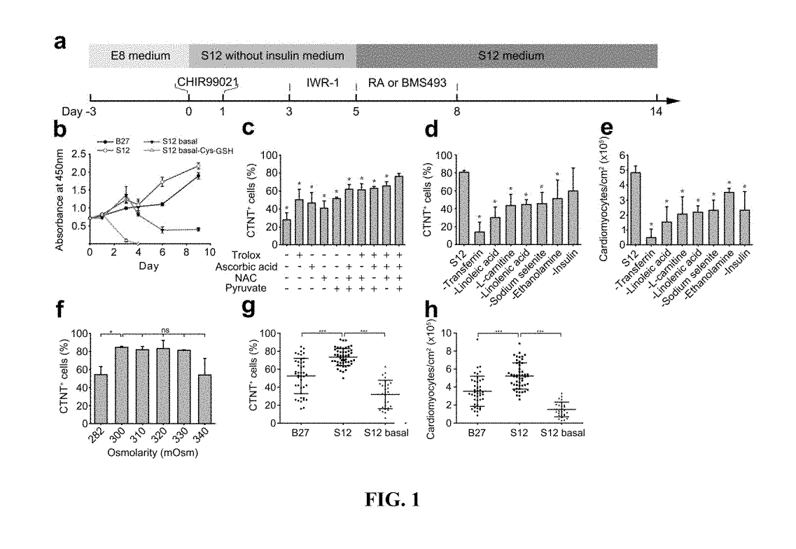

For small molecule-induced cardiac differentiation, hESC line H7 or hiPSCs were digested with Versene solution into single cells and seeded on VTN-NC-coated, 24-well plates at a density of 2.5-3.0×10⁵ cells/well. Y27632 (10 μM) was supplemented on the first day and withdrawn thereafter. Cultures were maintained in E8 medium for 2-3 days until they reached 90% confluence. To initiate cell differentiation (designated as day 0), the medium was changed to S12 medium without insulin. Cells were treated with 4-8 μM CHIR99021 (Tocris, 4423) for 1 day. Then, the medium was replaced with fresh medium without CHIR99021. At day 3, 5 μM IWR-1 (Sigma-Aldrich, 10161) was supplied with fresh medium for 2 days. From day 5, insulin-containing S12 medium was used. Thereafter, the medium was changed every other day (FIG. 1a).

In one aspect, insulin was removed or not included from the medium during the first 5 days of differentiation because inhibits early mesoderm differentiation of hESCs.¹³

For directed differentiation of atrial or ventricular myocytes, 1 μM RA (Sigma-Aldrich, R2625) or 1 μM BMS493 (Tocris, 3509) was added to the differentiating cultures during days 5-8 of differentiation as described previously⁹.

The formulations of E8 and S12 media are listed in Table 1 and Table 2, respectively.

In one embodiment, the formulations of the supplement and the medium are shown in Table 3.

TABLE 1
The formulation of E8 medium.

| | Supplier | Catalog number | Concentration | |
|---|---|---|---|---|
| DMEM/F12 | Gibco | 12500-062 | — | |
| Ascorbicacid-2-phosphate | Sigma | A8960 | 64 | mg/L |
| magnesium | | | | |
| Recombinant human | Repligen | 122-001 | 10.7 | mg/L |
| transferrin | Sweden AB | | | |
| Sodium selenium | Sigma | S5261 | 14 | μg/L |
| Insulin | Sigma | 91077C | 19.4 | mg/L |
| NaHCO3 | Sigma | S5761 | 543 | mg/L |
| NaCl | Sigma | S5886 | 1.2 | g/L |
| TGFβ-1 | Peprotech | 100-21 | 2 | μg/L |
| bFGF | Peprotech | 100-18B | 100 | μg/L |

Osmolarity = 340, pH 7.4, filtered, stored at 4° C., and used within 2 weeks

TABLE 2
The formulation of S12 medium.

| | | Catalog | | |
|---|---|---|---|---|
| | Supplier | number | Concentration | |
| RPMI 1640 | Life Technology | 31800-022 | — | |
| Recombinant human | Sigma | 91077C | 4 | mg/L |
| insulin | | | | |
| Recombinant human | Repligen | 122-001 | 5 | mg/L |
| transferrin | Sweden AB | | | |
| Sodium selenium | Sigma | S5261 | 16 | μg/L |
| Ethanolamine | Sigma | E0135 | 1 | mg/L |
| L-Carnitine | Sigma | C0283 | 2 | mg/L |
| hydrochloride | | | | |
| Linoleic acid | Sigma | L1012 | 1 | mg/L |
| Linolenic acid | Sigma | L2376 | 1 | mg/L |
| Methyl-β-cyclodextrin | Sigma | C4555 | 60 | mg/L |
| L-Ascorbic acid | Sigma | A4544 | 50 | mg/L |
| Trolox | Sigma | 238813 | 200 | μM |
| N-Acetyl-L-cysteine | Sigma | A7250 | 50 | μM |
| Sodium pyruvate | Sigma | P4562 | 1 | mM |

Osmolarity = 300, pH 7.2, filtered, stored at 4° C., and used within 2 weeks

TABLE 3
The formulations of the supplement and the medium in one
embodiment.

| | | | Supplement | | Final | |
|---|---|---|---|---|---|---|
| | | Catalog | concentration | | concentration | |
| Product name | Supplier | number | (in 10 ml) | | in medium | |
| Recombinant | Sigma | 91077C | 0.2 | mg/mL | 4 | mg/L |
| human insulin | | | | | | |
| Recombinant | Repligen | 122-001 | 0.25 | mg/mL | 5 | mg/L |
| human | Sweden | | | | | |
| transferrin | AB | | | | | |
| Sodium | Sigma | S5261 | 0.8 | μg/mL | 16 | μg/L |
| selenium | | | | | | |
| Ethanolamine | Sigma | E0135 | 0.05 | mg/mL | 1 | mg/L |
| L-Carnitine | Sigma | C0283 | 0.1 | mg/mL | 2 | mg/L |
| hydrochloride | | | | | | |
| Linolleic acid | Sigma | L1012 | 0.05 | mg/mL | 1 | mg/L |
| Linolenic acid | Sigma | L2376 | 0.05 | mg/mL | 1 | mg/L |
| Methyl-β- | Sigma | C4555 | 3 | mg/mL | 60 | mg/L |
| cyclodextrin | | | | | | |
| L-Ascorbic acid | Sigma | A4544 | 2.5 | mg/mL | 50 | mg/L |
| Trolox | Sigma | 238813 | 2.5 | mM | 50 | μM |
| N-Acetyl-L- | Sigma | A7250 | 2.5 | mM | 50 | μM |
| cysteine | | | | | | |
| Sodium pyruvate | Sigma | P4562 | 50 | mM | 1 | mM |

Note that linoleic acid and linolenic acid are two liposoluble components dissolved in the solvent, methyl-β-cyclodextrin.

Flow Cytometry.

Cardiomyocytes were digested into single cells with 0.25% trypsin-EDTA (Life Technology, 25200-072) at 37° C. for about 5 min, and washed with buffer B, which contains 0.5% bovine serum albumin (Sigma, A9418-50G) in phosphate-buffered saline (PBS). Then the cells were fixed with 4% paraformaldehyde at room temperature and washed with buffer A, which contains 0.5% bovine serum albumin and 0.1% saponin (Sigma-Aldrich, S7900) in PBS. The cells were then incubated with an anti-human CTNT primary antibody (R&D systems, MAB1874) for 40 min at 4° C. and washed with buffer A, followed by incubation with a goat anti-mouse FITC-conjugated secondary antibody (ZSGB-BIO, ZF-0312) for 40 min at 4° C. Data were collected on FACScalibur (Becton Dickinson) and analyzed with FlowJo software (Treestar).

RT-PCR and Quantitative Real-Time RT-PCR (QPCR).

Total RNA was isolated using RNeasy Plus Mini kit (Qiagen, 74134). One microgram of total RNA was reverse transcribed using the PrimeScript™ RT reagent kit with gDNA eraser (Takara, RR047A). RT-PCR was performed with 2×Taq Mix (CW-BIO, CW0682C) according to the manufacturer's instructions. PCR products were analyzed using 1.5% agarose gel electrophoresis. QPCR was performed in triplicate using the 2×QuantiFast SYBR Green I PCR Master Mix (Qiagen, 204057) on a Rotor Gene 6200 Real-Time PCR Machine (Corbett) with an annealing temperature of 60° C. The expression of each gene was normalized to GAPDH gene expression. The primer sequences are listed in Table 4 (primer sequences for RT-PCR) and Table 5 (primer sequences for QPCR).

TABLE 4
Primer sequences for RT-PCR.

| Gene | Forward primer | Reverse primer |
|---|---|---|
| POU5F1 | AACCTGGAGTTTGTGCCA | TGAACTTCACCTTCCCTC |
| | GGGTTT | CAACCA |
| T | TGTCCCAGGTGGCTTACA | GGTGTGCCAAAGTTGCCA |
| | GATGAA | ATACAC |
| WNT3A | AATGCCACTGCATCTTCC | TGGTGACAGTTCCTTGCT |
| | ACTGGT | GTCTGA |
| MESP1 | CACCGTCCCCGCTCCTTC | CGGCGTCAGTTGTCCCTT |
| | | GT |
| KDR | CCTCTACTCCAGTAAACC | TGTTCCCAGCATTTCACA |
| | TGATTGGG | CTATGG |
| CD31 | ATCATTTCTAGCGCATGG | ATTTGTGGAGGGCGAGGT |
| | CCTGGT | CATAGA |
| TBX5 | AAATGAAACCCAGCATAG | ACACTCAGCCTCACATCT |
| | GAGCTGGC | TACCCT |
| ISL1 | CACAAGCGTCTCGGGATT | AGTGGCAAGTCTTCCGAC |
| | GTGTTT | AA |
| NKX2.5 | GCGATTATGCAGCGTGCA | AACATAAATACGGGTGGG |
| | ATGAGT | TGCGTG |
| IRX4 | TTCCGTTCTGAAGCGTGG | TGAAGCAGGCAATTATTG |
| | TC | GTGT |
| TBX20 | GAAAGACCACACAGCCTC | TCAATGTCAGTGAGCCTG |
| | ATTGCT | GAGGAA |
| CTNT | TTCACCAAAGATCTGCTC | TTATTACTGGTGTGGAGT |
| | CTCGCT | GGGTGTGG |
| MLC2A | ACTGCCGAGACCGAGTAT | GCGATCCTTGAGGTTGTA |
| | G | GAGC |
| GAPDH | GAAATCCCATCACCATCT | GAGCCCCAGCCTTCTCCA |
| | TCCAGG | TG |

TABLE 5
Primer sequences for QPCR.

| Gene | Forward primer | Reverse primer |
|---|---|---|
| GAPDH | GAAATCCCATCACCATCT | GAGCCCCAGCCTTC |
| | TCCAGG | TCCATG |
| CTNT | TTCACCAAAGATCTGCTC | TTATTACTGGTGTG |
| | CTCGCT | GAGTGGGTGTGG |
| IRX4 | TTCCGTTCTGAAGCGTGG | TGAAGCAGGCAATT |
| | TC | ATTGGTGT |
| NR2F2 | GTGAGGGAGGTGAAAGAA | GGAAGAAAATCAAC |
| | CAGG | AACAACCGA |
| MLC2A | ACTGCCGAGACCGAGTAT | GCGATCCTTGAGGTT |
| | G | GTAGAGC |
| T | CAGTGGCAGTCTCAGGTT | CGCTACTGCAGGTGT |
| | AAGAAGGA | GAGCAA |
| POU5F1 | AACCTGGAGTTTGTGCCA | TGAACTTCACCTTCC |
| | GGGTTT | CTCCAACCA |
| MESP1 | CACCGTCCCCGCTCCTTC | CGGCGTCAGTTGTCC |
| | | CTTGT |
| ISL1 | TTGTACGGGATCAAATGC | AGGCCACACAGCGGA |
| | GCCAAG | AACA |
| NKX2.5 | ACCTCAACAGCTCCCTGA | ATAATCGCCGCCACA |
| | CTCT | AACTCTCC |
| TBX5 | TCCAGAAACTCAAGCTCA | TGGCAAAGGGATTAT |
| | CC | TCTCA |
| SOX2 | TTCACATGTCCCAGCACT | TCACATGTGTGAGAG |
| | ACCAGA | GGGCAGTGTGC |
| KLF4 | ACCCATCCTTCCTGCCCG | TTGGTAATGGAGCGG |
| | ATCAGA | CGGGACTTG |
| L-MYC | GCGAACCCAAGACCCAGG | CAGGGGGTCTGCTCG |
| | CCTGCTCC | CACCGTGATG |
| LIN28 | AGCCATATGGTAGCCTCA | TCAATTCTGTGCCTC |
| | TGTCCGC | CGGGAGCAGGGTAGG |

Cell Viability Assay.

The cell viability under different culture conditions was assessed with the CCK-8 kit according to the manufacturer's instructions (Dojindo). In brief, cells were seeded in a 24-well plate and differentiated. At each checkpoint, the cell culture medium was discarded and replaced with the reaction mixture (500 μL of RPMI 1640+50 μL of CCK-8 reagent for each well). After 40 min of incubation in a cell culture incubator, 110 μL of the reaction mixture from each well were transferred to a 96-well plate and the absorbance at 450 nm was acquired using a microplate reader (Perkin Elmer). Reaction mixture with unused medium was used as the blank control.

Protein Electrophoresis.

Sodium dodecyl sulfate-polyacrylamide gel electrophoresis (SDS-PAGE) was performed using the Invitrogen NuPAGE Bis-Tris Mini Gel Electrophoresis protocol. 4-12% NuPAGE Bis-Tris pre-cast gels (Invitrogen, NP0335PK2) and MES Running Buffer (Invitrogen, NP0002) were used to analyze the protein components of 100 μL of S12 and B27 media. The gels were stained with Coomassie brilliant blue.

Immunofluorescence.

Differentiated cultures were digested with 0.25% trypsin-EDTA, and the cells were plated on 0.1% gelatin-coated coverslips for 5 days to allow full attachment. The coverslips were washed with PBS and fixed in 4% paraformaldehyde for 20 min at 4° C. The fixed cells were then permeabilized with 0.1% Triton X-100 for 20 min at 4° C., and blocked with 10% goat serum (ZSGB-BIO, ZLI-9021) for 1 h at room temperature. Then, the cells were incubated overnight at 4° C. with the following primary antibodies: anti-human CTNT (R&D systems, MAB1874), mouse anti-human α-actinin (Sigma-Aldrich, A7811), mouse anti-human MHC (Abcam, ab15), goat anti-human α-SMA (Abcam, ab5694), mouse anti-human MLC2A (Synaptic Systems, 311011) and rabbit anti-human MLC2V (ProteinTech, 10906-1-AP). Goat anti-mouse 488- (ZSGB-BIO, ZF-0512) or 594-conjugated secondary antibody (ZSGB-BIO, ZF-0513), and goat anti-rabbit 488- (ZSGB-BIO, ZF-0511) or 594-conjugated secondary antibody (ZSGB-BIO, ZF-0516) were used as secondary antibodies. Nuclei were counterstained with 4′,6-diamidino-2-phenylindole (DAPI) (Sigma-Aldrich, D0542). Immunofluorescence images were visualized and recorded using an Olympus microscope system X51 or Olympus LSCM FV1000 (Olympus).

Differentiated cultures in T175 flasks were washed with PBS, fixed in 4% paraformaldehyde and permeabilized with 0.1% Triton X-100. The cells were first blocked with 10%0 goat serum (ZSGB-BIO, ZLI-9021) for 1 h. Then, the cells were incubated with the anti-human CTNT (R&D systems, MAB1874) for 1 h at 4° C., followed by incubation with Two-Step IHC Detection reagents (ZSGB-BIO, PV-6001) and DAB (ZSGB-BIO, ZLI-9018). Nuclei were counterstained with hematoxylin (ZSGB-BIO, ZLI-9609) for about 50 s. Immunostaining images were recorded by a Canon camera.

Single-Cell Preparation of hESC-Derived Cardiomyocytes.

The dissociation method used has been described previously⁹. Briefly, 25-30-day-old differentiated cardiomyocyte clusters were washed with a low Ca²⁺ solution, and then incubated in 1 mg/mL collagenase II (Life Technology, 17101-015) dissolved in a low Ca²⁺ solution for 30-40 min at 37° C. The isolated cells were suspended in S12 medium containing 10% FBS, transferred to 0.1% gelatin-coated glass coverslips, and then maintained in a 5% CO₂ incubator at 37° C.

Whole-Cell Patch Clamp Recording.

Three to eight days after dissociation, single adherent cells were selected for the patch clamp experiment. The spontaneously generated APs and ionic currents were recorded at 35±2° C., except for the Ca²⁺ currents, which were recorded at room temperature. Data were acquired at 10 kHz or 50 kHz, filtered at 2 kHz using Axon 200B and Digidata 1322 digitizer (Molecurlar Devices), and analyzed by the Clamfit 10 and Origin 8 software. Patch pipettes (2-4 MΩ resistance) filled with intracellular solutions were used. The formulations of intracellular and extracellular recording solutions for patch clamp are listed in Table 6 and Table 7.

TABLE 6
Intracellular solutions for patch clamp recording.

| | I_{Na} (mM) | I_{Ca} (mM) | I_{Kr} (mM) | AP (mM) |
|---|---|---|---|---|
| CsCl | 135 | 145 | | |
| NaCl | 10 | 5 | | |
| CaCl₂ | 2 | 2 | | |
| HEPES | 10 | 10 | 10 | 10 |
| MgATP | 5 | 5 | | |
| EGTA | 5 | 5 | 10 | 10 |
| KCl | | | 50 | 50 |
| K-aspartate | | | 80 | 80 |
| MgCl₂ 6H₂O | | | 1 | 1 |
| Na₂ATP | | | 3 | 3 |
| pH 7.2 | CsOH | CsOH | KOH | KOH |

TABLE 7
Extracellular solutions for patch clamp recording.

| | I_{Na} (mM) | I_{Ca} (mM) | I_{Kr} (mM) | AP(mM) |
|---|---|---|---|---|
| NaCl | 50 | | 140 | 140 |
| KCl | | | 5.4 | 5.4 |
| CaCl₂ H₂O | 1.8 | 5 | 1.8 | 1.8 |
| MgCl₂ 6H₂O | 1 | 1 | 1 | 1 |
| Glucose | 10 | 10 | 10 | 10 |
| HEPES | 10 | 10 | 10 | 10 |
| K-aspartate | 110 | | | |
| TEA-Cl | | 160 | | |
| 4-AP | | 2 | | |
| nifedipine | 0.001 | | 0.002 | |
| pH 7.4 | CsOH | CsOH | NaOH | NaOH |

Sodium current (I_{Na}) was recorded with 40-ms step pulses to a range between −80 mV and 80 mV with 10-mV increments, from a holding potential of −120 mV. Calcium current (I_{Ca}) was elicited by 300 ms pulses with 10-mV increments to a range from −70 mV to 60 mV, from a holding potential of −50 mV. To record the E-4031-sensitive current I_{Kr}, 1 μM E-4031 was applied to the extracellular solution to identify the I_{Kr}. The current was activated by 3 s pulses to a range between −40 mV and 20 mV with 5-mV increments, from a holding potential of −40 mV. Cell membrane capacitance (Cm) was measured by applying a 10-mV pulse from a holding potential of −70 mV. Current density was calculated by current peak amplitude divided by cell capacitance, and was used to plot the I-V curve.

Perforated whole-cell recording was used for pharmacological tests. Amphotericin B (240 μg/mL; Sigma, A2411) was added into the internal solution. The ventricular myocytes were selected and paced at 1 Hz by injecting a brief current pulse (0.1-0.3 nA). Drugs were applied from the lowest to highest concentration sequentially at 4 min intervals and APs were recorded at each concentration. The following parameters were analyzed: APD₉₀ and AP duration at 50% of repolarization (APD₅₀), APA, MDP and dV/dt. The formulations of the intracellular and extracellular recording solutions for patch clamp are listed in Table 6 and Table 7.

Stock solutions of E-4031 (Tocris, 1808) and isoproterenol (Sigma-Aldrich, 15627) were made in distilled water. Nifedipine (Sigma-Aldrich, N7634) was prepared in DMSO. The stock solutions were diluted with ≤0.1% DMSO in the external solution before use. Other chemicals were from Sigma-Aldrich.

Statistical Analysis.

Data are presented as mean±standard derivation (SD) or standard error of the mean (SEM). Statistical significance was calculated by Student's t-test (two tails). *P<0.05, ***P<0.001.

### Results

Albumin-Free and Chemically Defined Medium for hPSC Cardiac Differentiation.

To develop an albumin-free and chemically defined medium for cardiac differentiation, hESC line H7 was cultured in E8 medium for cell expansion¹⁰, and induced for cardiac differentiation with Wnt-related small molecules in the chemically defined RPMI 1640 medium, to evaluate the necessity of B27 components (FIG. 1a)². B27 has 20 components, among which there are four components (bovine serum albumin, catalase, superoxide dismutase and D-galactose) that originate from animals. Similarly to a previous report¹¹, removing albumin and catalase, superoxide dismutase and progesterone from B27 resulted in very low cardiac differentiation efficacy with cell growth arrest and moderate cell death. To prevent cell growth arrest and improve cardiac differentiation efficiency, 50 μg/mL recombinant human truncated vitronectin (VTN-NC) which could accelerate epithelial-mesenchymal transition (EMT) was added to the medium for the first three days of differentiation¹². However, when preparing the medium, DL-α-tocopherol acetate and DL-α-tocopherol, two vitamin E analogs, were insoluble in the medium without albumin. Hence, whether the water-soluble vitamin E analog, trolox, could support cardiac differentiation with B27 supplement without the first removed four ingredients, and DL-α-tocopherol acetate and DL-α-tocopherol, was tested. Meanwhile, linoleic acid and linolenic acid are two liposoluble components dissolved in the solvent, Methyl-3-cyclodextrin. Flow cytometry analysis (FACS) for CTNT positive cells indicated that with these six compounds removed, addition of trolox to the medium efficiently supported cardiac differentiation. Using this system, the effects of other components of B27 on cardiac differentiation were further evaluated. The results indicated that with the VTN-NC addition for the first three days, removing a total of 13 compounds from the medium did not significantly damage the cardiac differentiation efficacy or the cardiomyocyte yield (Table 8). In Table 8, the first column lists the 20 compounds of B27, vitronectin, and trolox. Columns a-l indicate the cardiac differentiation using the medium containing the compounds labeled in gray. The second row from the bottom indicates the cardiac differentiation efficacies, and the bottom row indicates the cardiomyocyte yields in each differentiation culture. “+” indicates the yield is similar to that of differentiation using B27-supplemented medium, and “−” indicates the yield is significantly lower than that of differentiation using B27-supplemented medium.

Based on these results we formulated a medium for hPSC cardiac differentiation, by supplementing RPMI 1640 medium with trolox and eight components. The eight components are recombinant human insulin (which inhibited early mesoderm differentiation of hESCs¹³ and removed from the medium during the first 5 days of differentiation), recombinant human transferrin (an iron carrier, which maintains cell homeostasis by regulating iron uptake^{14, 15}), sodium selenite (required for proper functioning of some antioxidant enzymes^{14, 15}), ethanolamine (a precursor for phospholipids synthesis, which is essential for the structure of the plasma membrane and cellular organelles¹⁵), L-carnitine hydrochloride (which plays an essential role in the transfer of long-chain fatty acids from the cytoplasm to the mitochondrial matrix for their oxidation, the predominant way of energy production during cardiac development¹⁶), linolenic acid and linoleic acid (which are essential fatty acids necessary to support optimal cardiovascular function¹⁷) dissolved Methyl-3-cyclodextrin (as a solvent). The RPMI1640 medium supplemented with the eight components was named S12 basal medium.

From the previous experiments, it was realized that from the six ingredients first depleted, albumin, superoxide dismutase, catalase, and the two vitamin E analogs (DL-α-tocopherol acetate and DL-α-tocopherol), have antioxidant properties. Therefore, antioxidant reagents may substitute the functions of the removed molecules, and support cell proliferation and cardiac differentiation of hPSCs in vitronectin-free medium. To test this hypothesis, whether antioxidants are critical for stem cell cardiac differentiation by differentiating cells in S12 basal medium with glutathione (a tripeptide consisting of glutamic acid, cysteine, and glycine) and L-cysteine (two components that have antioxidant functions)-depleted RPMI 1640 was first tested. In this antioxidant-free culture environment, cells died in three or four days (FIG. 1b). Then whether adding additional antioxidants to the S12 basal medium would support cardiac differentiation was tested. The results showed that the individual addition of the antioxidants, trolox, L-ascorbic acid, N-acetyl-L-cysteine (NAC), or sodium pyruvate to the S12 basal medium marginally improved the cardiac differentiation efficacy with relatively high inter-experimental variations. However, addition of any three of the four antioxidants improved the cardiac differentiation efficacy to over 60%, and addition of all four antioxidants together not only significantly increased the cardiac differentiation efficacy (76%; FIG. 1c), but also substantially prevented cell death in vitronectin-free medium (FIG. 1b). At this point, the S12 medium contained 12 supplemental components (S12) in RPMI 1640 medium, including two proteins, recombinant human insulin (4 mg/L) and recombinant human transferrin transferrin (5 mg/L), seven chemicals (L-ascorbic acid, trolox, NAC, sodium pyruvate, sodium selenite, ethanolamine, carnitine) and two fatty acids (linolenic acid and linoleic acid) dissolved in Methyl-β-cyclodextrin (as a solvent).

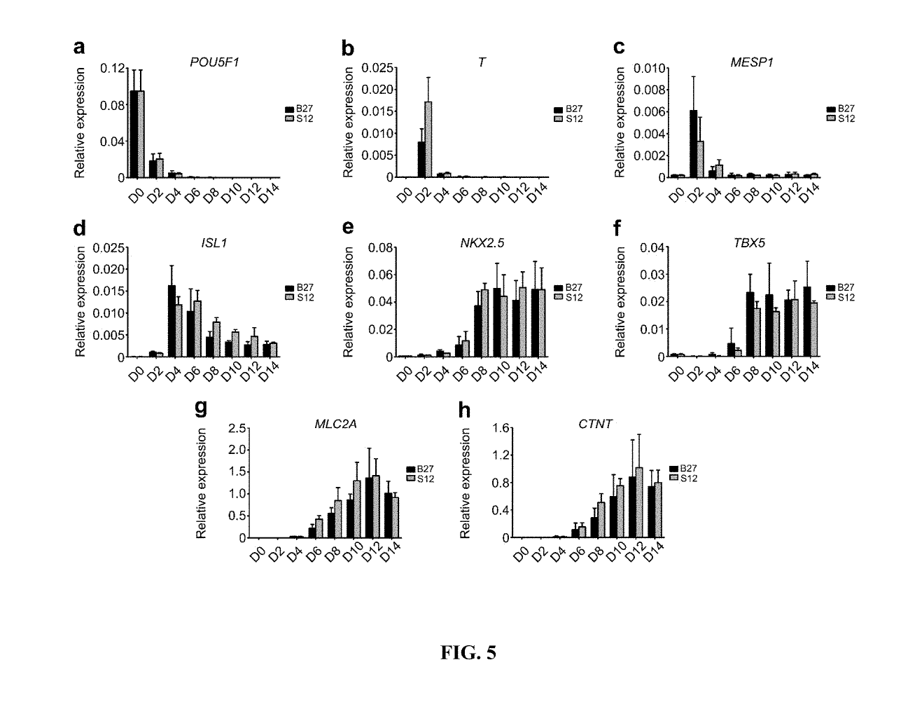

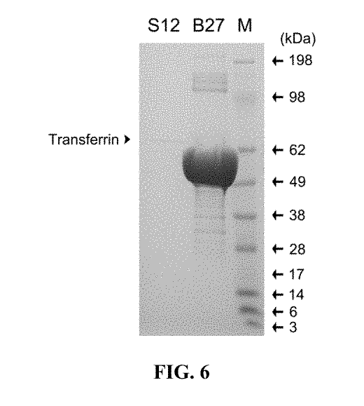

The expression levels of the pluripotent gene POU5F1, the mesodermal expressing genes Brachyury (T) and MESP1, and the cardiac-related genes ISL1, NKX2.5, TBX5, MLC2A and CTNT during the course of differentiation between cultures differentiated in the formulated medium versus the B27-supplemented medium were also compared by real-time quantitative reverse transcription polymerase chain reaction (RT-PCR). The results showed that cultures differentiated in these two media shared similar expression patterns of these genes (FIG. 5). Protein electrophoresis demonstrated that S12 medium contained significantly fewer proteins than the B27-supplemented medium (FIG. 6).

The necessity of the seven supplemented compounds other than the four antioxidants for cardiac differentiation was further evaluated. The results showed that individually removing six of these components, except for insulin, from S12 medium caused a statistically significantly decrease in the cardiac differentiation efficacy (FIG. 1d). The yield of cardiomyocytes differentiated in S12 medium was significantly higher than that in any of the media with component withdrawal (FIG. 1e). These results suggest that transferrin, linoleic acid, L-carnitine, linolenic acid, sodium selenite, ethanolamine and insulin are important for supporting efficient hPSC cardiac differentiation.

Osmolarity is important for stem cell culture in albumin-free conditions¹⁰. H7 cells were differentiated at different osmolarity levels, and the results indicated that the osmotic pressure range of 300-330 mOsm worked well for cardiac differentiation using S12 medium (FIG. 1f).

The reproducibility of cardiac differentiation is an important issue for large-scale production of human cardiomyocytes. In this study, the cardiac differentiation efficacies and the yields of cardiomyocytes of 42 individual cardiac differentiations were analyzed using three different batches of B27 supplement, 51 differentiations using S12 medium, and 27 differentiations using S12 basal medium in 24-well plates, conducted over 10 months. The statistics showed that the cardiac differentiation efficacy of cultures using S12 medium was significantly higher than that of the cultures using B27-supplemented medium (average 73.4% vs. 52.5%; FIG. 1g). More importantly, the standard deviation of the differentiation efficacy using S12 medium (8.6%) was substantially lower than when using B27-supplemented medium (19.2%), suggesting that the cardiac differentiation using S12 medium had a significantly higher reproducibility than that using B27-supplemented medium. Cardiomyocytes generated with S12 medium had an average density of 5.2×10⁵ cells/cm², which was 48.6% higher than that generated using B27-supplemented medium (3.5×10⁵ cells/cm²; FIG. 1h).

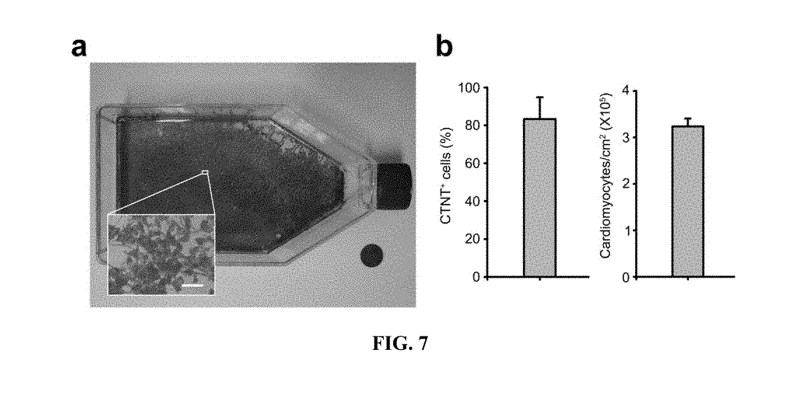

Large-scale generation of cardiomyocytes is a pre-requirement for their applications. H7 cells were differentiated in large-cell culture flasks. In three parallel differentiation sets with an average of 83.3% differentiation efficacy and a yield of 3.2×10⁵ cardiomyocytes/cm², an average of 5.6×10⁷ cardiomyocytes was generated from one T175 culture flask (FIG. 7). It was noticed that the cardiomyocyte yield per square centimeter of cultures in T175 flasks was lower than that of cultures in 24-well plates. This may reflect the difference in culture environments or the small sample number of T175 cultures. These differentiated cardiomyocytes were maintained in S12 medium for over 100 days with normal beating activity.

Generation of hiPSC Derived Atrial-Like and Ventricular-Like Cardiomyocytes in Albumin-Free and Chemically Defined Culture Conditions.

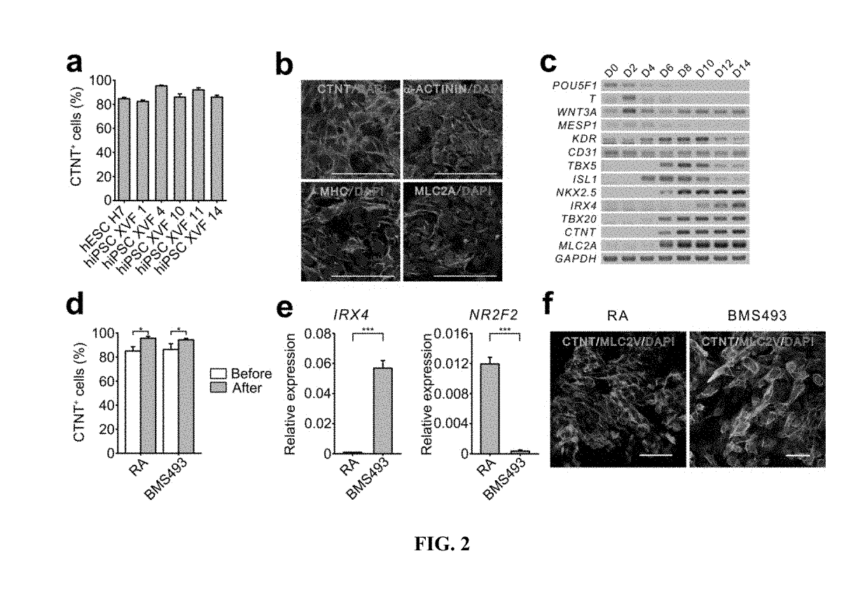

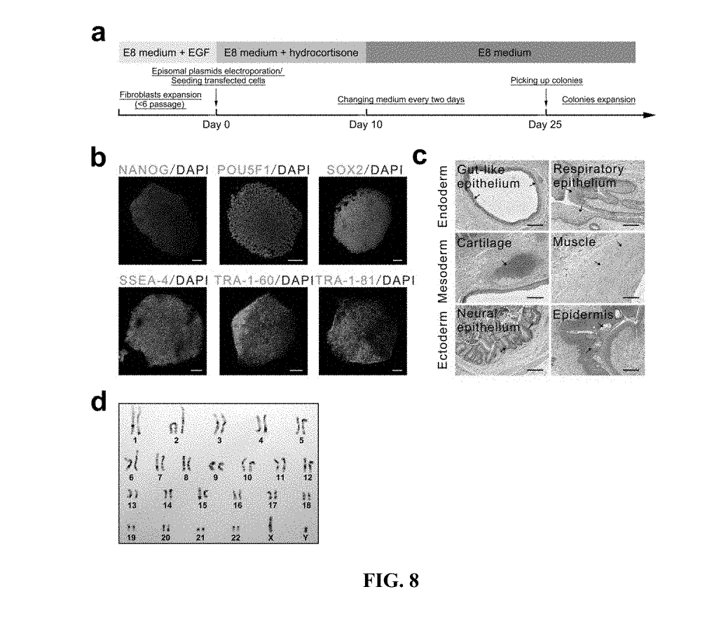

Using recombinant protein-based E8 medium (by substituting human holo-transferrin with Saccharomyces cerevisiae-expressed recombinant human holo-transferrin)¹⁰, recombinant human vitronectin as substrate, episomal plasmid-based vectors expressing Yamanaka factors, and recombinant enzymatic passaging, 24 pluripotent hiPSC lines were generated in chemically defined and albumin-free conditions^{10, 18} (FIG. 8a). These hiPSC lines uniformly expressed POU5F1, NANOG, SOX2, and other pluripotent genes, and formed teratoma with tissues of three germ layers (FIG. 8b and FIG. 8c). Genetic examination indicated that these hiPSC lines have a normal karyotype (FIG. 8d). Five of the hiPSC lines were tested for cardiac differentiation in S12 medium. FACS analysis of CTNT-expressing cells indicated that all five lines differentiated efficiently into cardiomyocytes (FIG. 2a). Fluorescent immunostaining and RT-PCR demonstrated that these differentiated cardiomyocytes expressed mesodermal and cardiac genes during the course of differentiation (FIG. 2b and FIG. 2c).

To directly differentiate hPSCs into highly homogeneous atrial- and ventricular-like cardiomyocytes, one of the hiPSC lines, xeno and virus free (XVF) 4, was differentiated in S12 medium, and treated with retinoic acid (RA) for atrial myocyte differentiation or the RA inhibitor, BMS493, for ventricular myocyte differentiation, as we have reported previously⁹. FACS analysis of CTNT-expressing cells indicated that the cardiac differentiation efficacies were over 80% in both of differentially treated cultures (FIG. 2d). Real-time quantitative RT-PCR indicated that the expression levels of the ventricular-specific gene, IRX4, were significantly higher in the BMS493-treated cultures than in the RA-treated cultures. On the other hand, the expression levels of the atrial-specific gene, NR2F2¹⁹, were significantly higher in the RA-treated cultures than in the BMS493-treated cultures (FIG. 2e). Double fluorescent immunostaining of CTNT and the mature ventricular myocyte marker, MLC2V⁹, in 60-day-old cultures indicated that MLC2V was widely expressed in cardiomyocytes of BMS493-treated cultures, but not in those of RA-treated cultures (FIG. 2f).

The cardiomyocytes of the differentially treated cultures were further enriched using a metabolic selection strategy by replacing glucose with DL-lactate in RPMI 1640 medium from day 14 of differentiation²⁰. FACS analysis showed that only 2 days of glucose deprivation enriched cardiomyocytes from 85% to 95.7% in RA-treated cultures, and from 86.3% to 94.3% in BMS493-treated cultures (FIG. 2d).

Electrophysiological Characterization of hPSC-Derived Atrial- and Ventricular-Like Cardiomyocytes.

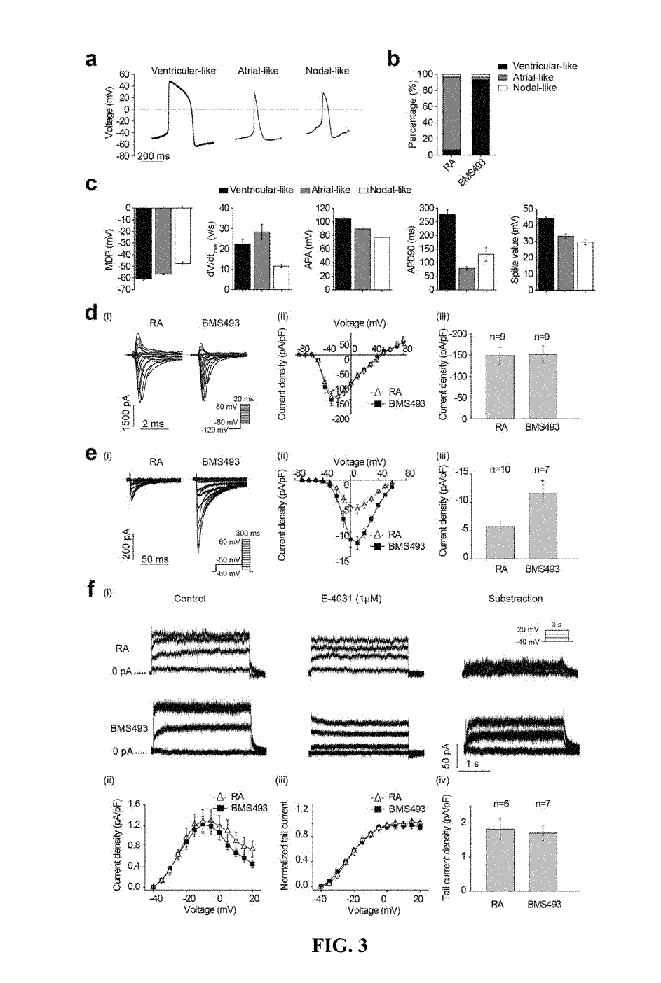

Action potential (AP) and ion channel activities, which are critical for normal functions of cardiomyocytes, were investigated using whole-cell patch clamp technology on single cardiomyocytes. The classification of nodal-, atrial- and ventricular-like APs was based on the AP properties. Particularly, nodal-like AP has a slower maximal upstroke velocity (dV/dt), smaller action potential amplitude (APA), depolarized maximum diastolic potential (MDP), and faster beating rate than those of atrial- and ventricular-like APs. An AP duration at 90%0, of repolarization (APD₉₀) value of 150 ms was used to classify atrial- and ventricular-like APs, because of the significant plateau phase of ventricular AP^{21, 22}. The results showed that even though all three AP types were recorded from the cultures (FIG. 3a), 90.3% (n=31) of the APs recorded in cardiomyocytes of the RA-induced culture were atrial-like APs, and 93.3% (n=30) of those in the BMS493-treated culture were ventricular-like APs (FIG. 3b). These results demonstrated that the cardiomyocytes in the RA-treated cultures are highly homogenous atrial-like cardiomyocytes, and those in the BMS493-treated cultures were highly homogenous ventricular-like cardiomyocytes. The parameters of the three AP types recorded were also analyzed (FIG. 3c).

Sodium current (I_{Na}) and calcium current (I_{Ca}), and E-4031-sensitive current (I_{Kr}) of atrial- and ventricular-like cardiomyocytes were also investigated. The membrane capacitance of ventricular-like cardiomyocytes (35.1±2.3 pF, n=23) was larger than that of atrial-like cardiomyocytes (25.3±2.0 pF, n=26). The maximum I_{Na} densities in ventricular-like cardiomyocytes and atrial-like cardiomyocytes were −151.7±20.0 pA/pF (n=9) and −148.5±19.7 pA/pF (n=9; FIG. 3d), respectively. For I_{Ca}, the peak current density of ventricular-like cardiomyocytes was larger than that of atrial-like cardiomyocytes (−11.5±1.6 pA/pF, n=7 vs. −5.7±0.9 pA/pF, n=10; FIG. 3e). To measure I_{Kr}, 1 μM E-4031 was used to isolate the current. I_{Kr} were present in all recorded cells of ventricular-like cardiomyocytes (n=7) and atrial-like cardiomyocytes (n=6). The peak tail current densities of I_{Kr} in ventricular-like cardiomyocytes and atrial-like cardiomyocytes were 1.7±0.2 and 1.8±0.3 pA/pF, respectively (FIG. 3f). In summary, in the stem cell-derived atrial- and ventricular-like cardiomyocytes, significant differences in the AP durations and amplitudes of the I_{Ca}, but not in the amplitudes of the I_{Na} and I_{Kr}, were observed.

Exploration of Cardiac Safety Studies Using the hPSC Derived Ventricular-Like Cardiomyocytes.

hPSC-CMs provide a unique opportunity for cardiac safety assessment in drug discovery. Because of the regulatory requirements of ICH S7B for analyzing the risk of drug-induced torsades de pointes (TdP), the life-threatening ventricular arrhythmia²³, the most relevant subtype of cardiomyocytes for cardio-toxicity analysis is the ventricular-like cardiomyocytes²⁴.

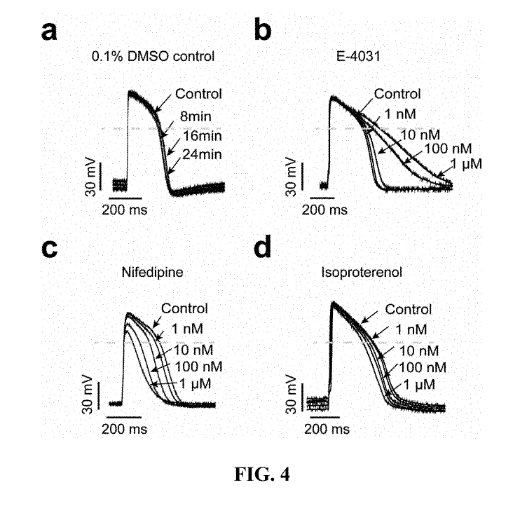

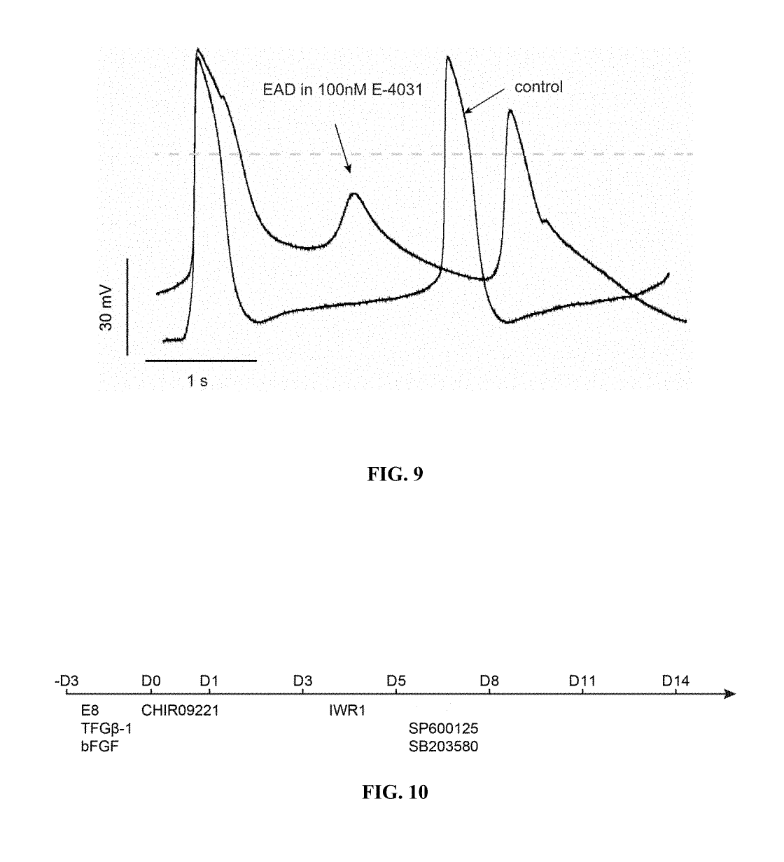

In this study, three reference compounds (E-4031, nifedipine, and isoproterenol) were tested for their effect on APs recorded from ventricular-like cardiomyocytes using whole-cell patch clamp techniques. The results showed that extracellular solution with 0.1% DMSO had no effect on evoked ventricular-like AP in a 20 min recording period. E-4031, a hERG channel blocker, at 100 nM induced early afterdepolarization (EAD) in spontaneously beating cells (FIG. 9). When ventricular-like cardiomyocytes were evoked with a 1 Hz stimulation (0.1-0.3 nA), E-4031 prolonged the AP duration in a dose-dependent manner. E-4031 (10 nM) had minor effects on the AP, while APD₉₀ was prolonged to 169.6±13.4% at 100 nM and 195.6±12.3% at 1000 nM. However, there was no significant change observed in APA and dV/dt (FIG. 4b). Nifedipine, an inhibitor of L-type Ca²⁺ channels, shortened the APD₉₀ to 73.6±2.7% at 10 nM, 60.8±4.3% at 100 nM, and 47.1±6.1% at 1 μM, with minor reductions in APA and dV/dt at 1000 nM. Isoproterenol, a 3-adrenergic agonist that increases the heart rate, had no significant effects on the parameters of AP except for a minor reduction in the APD₉₀ (FIG. 4 and Table 9). In Table 9, data were calculated as the percentage of those of APs collected from untreated cardiomyocytes, and was presented as mean±SEM. MDP: maximal depolarized potential; APD₅₀ and APD₉₀: action potential duration at 50% and 90% of repolarization; APA: action potential amplitude; dV/dt: maximal upstroke velocity.

TABLE 9
The statistics of the pharmacological effects on evoked AP.

| | n | MDP | APD₅₀ | APD₉₀ | APA | dV/dT |
|---|---|---|---|---|---|---|
| Control | | | | | | |
| 4 min | 5 | 101.1 ± 1.0 | 98.5 ± 2.1 | 98.7 ± 1.5 | 100.2 ± 0.7 | 97.1 ± 4.8 |
| 8 min | 5 | 100.2 ± 2.7 | 97.1 ± 1.9 | 97.4 ± 1.8 | 94.8 ± 1.4 | 94.8 ± 4.8 |
| 12 min | 5 | 100.2 ± 1.5 | 95.1 ± 3.4 | 95.0 ± 2.7 | 99.9 ± 1.4 | 95.9 ± 7.5 |
| 16 min | 4 | 101.5 ± 1.5 | 100.6 ± 1.2 | 99.2 ± 1.1 | 99.8 ± 1.8 | 101.5 ± 7.7 |
| E-4031 | | | | | | |
| 1 nM | 4 | 101.9 ± 1.2 | 103.8 ± 3.8 | 102.1 ± 3.1 | 102.9 ± 1.1 | 101.2 ± 8.0 |
| 10 nM | 4 | 101.5 ± 1.7 | 110.1 ± 5.4 | 112.1 ± 5.0 | 103.8 ± 1.5 | 112.4 ± 5.5 |
| 100 nM | 4 | 99.6 ± 2.9 | 144.0 ± 6.7* | 169.6 ± 13.4* | 103.0 ± 2.3 | 111.4 ± 9.6 |
| 1000 nM | 4 | 92.4 ± 2.8 | 151.1 ± 7.5* | 195.6 ± 12.3* | 95.6 ± 4.0 | 95.6 ± 7.4 |
| Nifedipine | | | | | | |
| 1 nM | 5 | 99.7 ± 2.7 | 92.7 ± 2.1 | 94.6 ± 2.0 | 96.7 ± 2.7 | 90.6 ± 4.1 |
| 10 nM | 5 | 103.5 ± 1.1 | 70.1 ± 2.9* | 73.6 ± 2.7* | 96.2 ± 0.7 | 96.4 ± 5.9 |
| 100 nM | 5 | 100.8 ± 2.9 | 55.3 ± 4.3* | 60.8 ± 4.3* | 88.3 ± 2.4 | 86.1 ± 8.8 |
| 1000 nM | 5 | 100.8 ± 3.1 | 36.0 ± 3.3* | 47.1 ± 6.1* | 73.8 ± 4.4* | 60.0 ± 11.3* |
| Isoproterenol | | | | | | |
| 1 nM | 4 | 101.2 ± 1.5 | 99.1 ± 0.7 | 97.3 ± 1.7 | 99.9 ± 1.7 | 99.9 ± 3.0 |
| 10 nM | 4 | 102.8 ± 2.3 | 93.0 ± 1.6 | 92.0 ± 2.5 | 101.7 ± 1.6 | 108.1 ± 1.8 |
| 100 nM | 4 | 104.8 ± 3.7 | 90.7 ± 1.8 | 90.8 ± 1.8 | 102.1 ± 2.5 | 105.6 ± 2.5 |
| 1000 nM | 4 | 101.8 ± 5.0 | 87.0 ± 1.6* | 87.3 ± 1.6* | 99.0 ± 5.2 | 105.3 ± 3.1 |

The results of the effects of E4031 and nifedipine on the AP of our homogenous ventricular-like cardiomyocytes are similar to those reported in previous studies on hPSC-derived ventricular-like cardiomyocytes generated with un-directed differentiation protocols^{25, 26}.

### Discussion

In this example, it was found that maintaining a proper level of reactive oxygen species (ROS) in the culture is critical for proliferation and efficient cardiac differentiation of hPSCs. ROS is produced naturally and continuously by metabolic and other physiological processes in cells. Deficiency in the antioxidant system in culture medium results in spontaneous ROS accumulation in cells, and results in oxidative stress, leading to permanent growth arrest and cell apoptosis^{27, 28}. Among the four antioxidants present in the S12 medium, ascorbic acid and trolox are antioxidants that react with free radicals, such as peroxyle radicals and singlet molecular oxygen (¹O₂), in aqueous compartments and biological lipid phases²⁹. NAC is not only a precursor of glutathione, but it also interacts directly with ROS and nitrogen species as a scavenger of oxygen free radicals³⁰. Pyruvate is a scavenger of hydrogen peroxide through a non-enzymatic reaction in cell culture medium³¹. E8 medium showed for the first time that albumin, previously considered an essential cell culture medium compound, could be eliminated from the medium for supporting long-term hPSC culture¹⁰. The results further demonstrated that by modulating the antioxidant levels in the medium, long-term culture of differentiated cells in albumin-free conditions could be achieved. These studies provided new insights into strategies for developing albumin-free media in the field of cell culture research.

Even though a report on transplantation of hPSC-CMs into an infarct myocardium primate model has demonstrated the potentials of cell therapy for heart failure³², the concern of arrhythmias caused by the transplanted cells has been raised³³. Without directed differentiation, atrial myocytes, ventricular myocytes and nodal cells were spontaneously differentiated³⁴. It has been demonstrated that transplanting a heterogeneous myocyte population to the left ventricle provoked an ectopic heart rhythm^{35, 36}. Using a highly homogeneous ventricular myocyte population generated under albumin-free and chemically defined conditions in cell-based transplantation therapies for heart infarction has the potential to not only reduce the risk of arrhythmias, but also ease the regulatory concerns of bio-safety issues.

hPSC-derived ventricular myocytes provide a novel, time- and cost-efficient, proarrhythmic risk assessment paradigm for cardio-toxicity analysis of drugs, and is currently under investigation for the possibility of being used as a supplementary technology for drug cardiac safety evaluation by the relevant government administration, academia and pharmaceutical industry³⁷. Because hPSC-derived ventricular-like cardiomyocytes possess currents expected of adult ventricular myocytes^{26, 38}, using these cells for cardiac safety assessments can not only avoid specie differences, but can also obtain the integrated, cell physiological drug response, which cannot be obtained from the traditional single ion channel transgenic cellular models²⁴. Generation of highly homogeneous hPSC-derived ventricular-like cardiomyocytes under albumin-free and chemically defined conditions is a pre-requirement for the development of high-throughput drug screening technologies, such as automatic patch clamp, for early preclinical safety assessment of drug discovery compounds²³.

In summary, eliminating albumin from the medium offers several advantages. Firstly, it eradicates the batch-to-batch variability of albumin, which significantly increases the reproducibility of the cardiac differentiation process. More ventricular-like or atrial-like cardiomyocytes can be obtained by large-scale cardiac differentiation. Secondly, it excludes the interference caused by the interaction between albumin and the tested molecules in cell-based analyses³⁹. So it provided a platform to produce mature ventricular-like or atrial-like cardiomyocytes by exploring different potential factors. Most importantly, it reduces the risk of potential pathogen contamination and cell immunogenicity in clinical applications of these cells. This albumin-free and chemically defined medium will facilitate both research and clinical applications of hPSC-CMs in the future.

### REFERENCES

1. Yang L et al. Human cardiovascular progenitor cells develop from a kdr+ embryonic-stem-cell-derived population. Nature. 2008; 453:524-528.
2. Lian X et al. Robust cardiomyocyte differentiation from human pluripotent stem cells via temporal modulation of canonical wnt signaling. Proceedings of the National Academy of Sciences of the United States of America. 2012; 109:E1848-1857.
3. Takahashi K et al. Induction of pluripotent stem cells from adult human fibroblasts by defined factors. Cell. 2007; 131:861-872.
4. Thomson J A et al. Embryonic stem cell lines derived from human blastocysts. Science. 1998; 282:1145-1147.
5. Stacey G N et al. The development of ‘feeder’ cells for the preparation of clinical grade hes cell lines: Challenges and solutions. Journal of biotechnology. 2006, 125:583-588.
6. Ng E S et al. A protocol describing the use of a recombinant protein-based, animal product-free medium (apel) for human embryonic stem cell differentiation as spin embryoid bodies. Nature protocols. 2008; 3:768-776.
7. Burridge P W et al. Chemically defined generation of human cardiomyocytes. Nature methods. 2014.
8. Kobayashi K. Summary of recombinant human serum albumin development. Biologicals: journal of the International Association of Biological Standardization. 2006; 34:55-59.
9. Zhang Q et al. Direct differentiation of atrial and ventricular myocytes from human embryonic stem cells by alternating retinoid signals. Cell Res. 2011; 21:579-587.
10. Chen G et al. Chemically defined conditions for human ipsc derivation and culture. Nature methods. 2011; 8:424-429.
11. Minami I et al. A small molecule that promotes cardiac differentiation of human pluripotent stem cells under defined, cytokine- and xeno-free conditions. Cell reports. 2012; 2:1448-1460.
12. Zhang J et al. Extracellular matrix promotes highly efficient cardiac differentiation of human pluripotent stem cells: The matrix sandwich method. Circulation research. 2012; 111:1125-1136.
13. Xu X Q et al. Chemically defined medium supporting cardiomyocyte differentiation of human embryonic stem cells. Differentiation. 2008; 76:958-970.
14. Gstraunthaler G. Alternatives to the use of fetal bovine serum: Serum-free cell culture. Altex-Altern Tierexp. 2003; 20:275-281.
15. Barnes D, Sato G. Serum-free cell culture: A unifying approach. Cell. 1980; 22:649-655.
16. Longo N et al. Disorders of carnitine transport and the carnitine cycle. Am J Med Genet C. 2006; 142C:77-85.
17. Duda M K et al. Omega-3 polyunsaturated fatty acid supplementation for the treatment of heart failure: Mechanisms and clinical potential. Cardiovasc Res. 2009; 84:33-41.
18. Okita K et al. A more efficient method to generate integration-free human ips cells. Nature methods. 2011; 8:409-412.
19. Wu S P et al. Atrial identity is determined by a coup-tfii regulatory network. Developmental cell. 2013; 25:417-426.
20. Tohyama S et al. Distinct metabolic flow enables large-scale purification of mouse and human pluripotent stem cell-derived cardiomyocytes. Cell stem cell. 2013; 12:127-137.
21. Peng S et al. The action potential and comparative pharmacology of stem cell-derived human cardiomyocytes. Journal of pharmacological and toxicological methods. 2010; 61:277-286.
22. Honda et al. Electrophysiological characterization of cardiomyocytes derived from human induced pluripotent stem cells. Journal of pharmacological sciences. 2011; 117:149-159.
23. International conference on harmonisation; guidance on s7b nonclinical evaluation of the potential for delayed ventricular repolarization (qt interval prolongation) by human pharmaceuticals; availability. Notice. Federal register. 2005; 70:61133-61134.
24. Mandenius C F et al. Cardiotoxicity testing using pluripotent stem cell-derived human cardiomyocytes and state-of-the-art bioanalytics: A review. J Appl Toxicol. 2011; 31:191-205.
25. Gibson J K et al. Human stem cell-derived cardiomyocytes detect drug-mediated changes in action potentials and ion currents. Journal of pharmacological and toxicological methods. 2014; 70:255-267.
26. Ma J et al. High purity human-induced pluripotent stem cell-derived cardiomyocytes: Electrophysiological properties of action potentials and ionic currents. American journal of physiology. Heart and circulatory physiology. 2011; 301:H2006-2017.
27. Davies K J. The broad spectrum of responses to oxidants in proliferating cells: A new paradigm for oxidative stress. IUBMB life. 1999, 48:41-47.
28. Morel Y, Barouki R. Repression of gene expression by oxidative stress. The Biochemical journal. 1999; 342 Pt 3:481-496.
29. Sies H, Stahl W. Vitamins e and c, beta-carotene, and other carotenoids as antioxidants. The American journal of clinical nutrition. 1995; 62:1315S-1321S.
30. Zafarullah M et al. Molecular mechanisms of n-acetylcysteine actions. Cellular and molecular life sciences: CMLS. 2003; 60:6-20.
31. Babich H et al. Choice of dmem, formulated with or without pyruvate, plays an important role in assessing the in vitro cytotoxicity of oxidants and prooxidant nutraceuticals. In vitro cellular & developmental biology. Animal. 2009; 45:226-233.
32. Chong J J et al. Human embryonic-stem-cell-derived cardiomyocytes regenerate non-human primate hearts. Nature. 2014.
33. Anderson M E et al. Embryonic stem cell-derived cardiac myocytes are not ready for human trials. Circulation research. 2014; 115:335-338.
34. He J Q et al. Human embryonic stem cells develop into multiple types of cardiac myocytes: Action potential characterization. Circulation research. 2003; 93:32-39.
35. Kehat I et al. Electromechanical integration of cardiomyocytes derived from human embryonic stem cells. Nature biotechnology. 2004; 22:1282-1289.
36. Chen H S et al. Electrophysiological challenges of cell-based myocardial repair. Circulation. 2009; 120:2496-2508.
37. Cavero I, Holzgrefe H. Comprehensive in vitro proarrhythmia assay, a novel in vitro/in silico paradigm to detect ventricular proarrhythmic liability: A visionary 21st century initiative. Expert opinion on drug safety. 2014; 13:745-758.
38. Hoekstra M et al. Induced pluripotent stem cell derived cardiomyocytes as models for cardiac arrhythrnias. Front Physiol. 2012; 3.
39. Francis G L. Albumin and mammalian cell culture: Implications for biotechnology applications. Cytotechnology. 2010; 62:1-16.

### Example 2

### Role of SAPK/JNK Pathway and p38 Pathway in Stem Cell Differentiation in a Chemically Defined Albumin-Free Culture

In this example, the roles of SAPK/JNK pathway and p38 MAPK pathway in regulating ventricular myocyte specification of differentiated human pluripotent stem cells were investigated under the chemically defined, albumin-free culture conditions. See Derynck and Zhang, Nature 425: 577-84 (2003); Miyazono et al., J Biochem. 147: 35-51 (2010).

As described in FIG. 10, human pluripotent stem cell line H7 was differentiated into cardiomyocytes with subsequently treatments with CHIR09221 and IWR1. During day 5-8 of differentiation, the time window in which the subtypes of stem cell differentiated cardiomyocytes were specified, SAPK/JNK pathway or p38 MAPK pathway was blocked with their small molecule inhibitor SP600125 (for SAPK/JNK) or SB203580 (for P38 pathway) respectively. See Bennett et al., PNAS 98: 13681-86 (2001); Lali et al., J. Biol. Chem. 275: 7395-402 (2000).

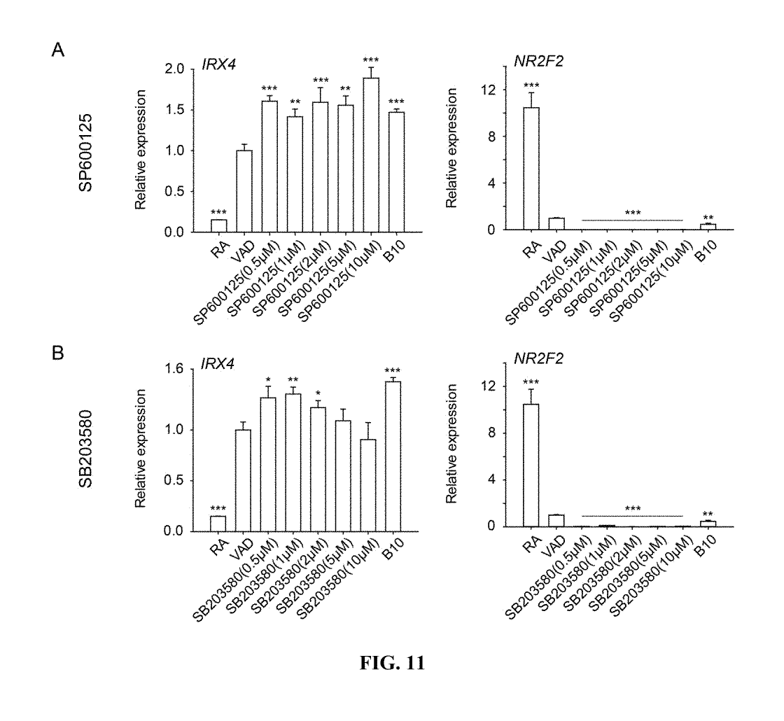

At D14, quantitative real time reverse transcript polymer chain reaction analyses were used to examine the expression levels of ventricular specific gene IRX-4⁵, and atrial myocytes specific gene NR2F2. See Zhang et al., Cell Res. 21: 579-87 (2011); Devalla et al., EMBO Mol. Med. 7: 394-410 (2015). The results, showed in FIG. 11, indicated that comparing with the un-treated group, inhibitions of either SAPK/JNK or P38 pathway elevated the expression levels of ventricular specific gene IRX4, and repressed the expression of atrial specific gene NR2F2. These results indicated that inhibitions of SAPK/JNK or P38 pathways promote ventricular versus atrial myocyte specification, and small molecule inhibitor of these two pathways can be used to promote ventricular myocyte differentiation of stem cells.

Citation of the above publications or documents is not intended as an admission that any of the foregoing is pertinent prior art, nor does it constitute any admission as to the contents or date of these publications or documents.

1. A cell culture medium supplement comprising at least one antioxidant or at least two different antioxidants that substitute(s) the function of albumin in a cell culture medium, wherein said cell culture medium supplement is configured to be combined with a basal culture medium to form a substantially albumin-free cell culture medium.

2. A cell culture medium supplement comprising at least one antioxidant or at least two different antioxidants selected from the group consisting of:
a) ascorbic acid, ascorbate, or a salt or an ester thereof,
b) a water-soluble analog of vitamin E,
c) N-acetyl-cysteine or glutathione, or a salt or an ester thereof,
d) pyruvic acid, pyruvate, or a salt or an ester thereof,
e) a catalase,
f) a superoxide dismutase,
g) a thiol, such as 2-mercaptoethanol or 1-thioglycerol,
h) a metallothione,
i) a thioredoxin,
j) lipoic acid or a salt or an ester thereof,
k) uric acid or a salt or an ester thereof,
l) a carotene,
m) melatonin,
n) probucol,
o) dimethylthiourea, and
p) resveratrol,
wherein said cell culture medium supplement is configured to be combined with a basal culture medium to form a substantially albumin-free cell culture medium.

3-8. (canceled)

9. The cell culture medium supplement of claim 1, which further comprises an iron carrier.

10. The cell culture medium supplement of claim 1, which further comprises a polypeptide, which polypeptide is optionally the iron carrier.

11-16. (canceled)

17. The cell culture medium supplement of claim 1, which further comprises a water-soluble selenium compound.

18-19. (canceled)

20. The cell culture medium supplement of claim 1, which further comprises a C₁₋₈ alkanolamine.

21-26. (canceled)

27. The cell culture medium supplement of claim 1, which further comprises a fatty acid, such as linoleic acid and linolenic acid or a combination thereof, which fatty acid is optionally dissolved in the solvent methyl-β-cyclodextrin.

28-36. (canceled)

37. A substantially albumin-free cell culture medium, which comprises a substantially albumin-free basal culture medium and a cell culture medium supplement of claim 1.

38-49. (canceled)

50. The cell culture medium of claim 37, which is configured to support growth, differentiation, and/or maintenance of a cell.

51-59. (canceled)

60. A kit which comprises a cell culture medium of claim 37.

61. (canceled)

62. The kit of claim 60, which further comprises a substance that initiates, directs and/or supports differentiation and/or maintenance of a stem cell, a progenitor cell, or a precursor cell.

63-76. (canceled)

77. A method for growing, differentiating and/or maintaining a cell, which method comprises contacting a cell with a substantially albumin-free cell culture medium of claim 37.

78-82. (canceled)

83. The method of claim 77, wherein the cell is a stem cell.

84-93. (canceled)

94. The method of claim 83, which further comprises contacting a stem cell or a mesodermal cell with a substance to initiate, direct and/or support differentiation of the stem cell or the mesodermal cell into a cardiomyocyte, e.g., a ventricular cardiomyocyte and/or an atrial cardiomyocyte.

95-113. (canceled)

114. A cell grown, differentiated and/or maintained by the method of claim 77.

115. A cardiomyocyte produced by the method of claim 94.

116. A cardiomyocyte of claim 115, which has elevated expression level of a cardiomyocyte specific gene, embryonic cardiomyocyte-like action potentials (AP) and/or Ca²⁺ spark pattern typical of a cardiomyocyte.

117-120. (canceled)

121. A pharmaceutical composition, which pharmaceutical composition comprises an effective amount of the cells grown, differentiated and/or maintained by the method of claim 77, and a pharmaceutically acceptable carrier or excipient.

122. (canceled)

123. A method for treating a disease or disorder in a subject, which method comprises administering, to a subject to which such treatment is needed or desirable, an effective amount of the pharmaceutical composition of claim 121.

124-125. (canceled)

126. A method for identifying a modulator of a cell, which method comprises:
1) contacting a cell of claim 114 with a modulator candidate and measuring the effect of the modulator candidate on a property of the cell; and
2) measuring the property of the cell not contacted with the modulator candidate, whereby the property of the cell contacted with the modulator candidate is different from that of the cell not contacted with the modulator candidate identifies the modulator candidate as a modulator of the property of the cell.

127. (canceled)
